SEQUENCE LISTING (42 sequences)

SEQ ID NO: 1 - protein, 33 aa, Artificial Sequence
  Description of Artificial Sequence Synthetic polypeptide
KKKKYALADASLKMADPNRFRGKDLPVLDQLLE

SEQ ID NO: 2 - protein, 5 aa, Artificial Sequence
  Description of Artificial Sequence Synthetic peptide
ADPNR

SEQ ID NO: 3 - protein, 127 aa, Artificial Sequence
  Description of Artificial Sequence Synthetic polypeptide
EVQLVESGGGVVQPGRSQRLSCAASGFTFRRYAIHWVRQAPGKGLEWVAVISNDGSNKFYAESVEGRFTVSRDNSMNTVYLQMNGLRAEDTAVYYCARAPEYCSGGSCYSQYYFDYWGRGTLVTVSS

SEQ ID NO: 4 - protein, 120 aa, Artificial Sequence
  Description of Artificial Sequence Synthetic polypeptide
EVQLVESGGGLVQPGGSLRLSCAASGFTFSDYWMSWVRQAPGKGLEWVANIKYDGSEKYYVDSVKGRFTTSRDNAKNSLYLQMNSLRAEDTAVYYCAGLLWFGEKAFDIWGQGTMVTVSS

SEQ ID NO: 5 - protein, 127 aa, Artificial Sequence
  Description of Artificial Sequence Synthetic polypeptide
EVQLVESGGGVVQPGRSLRLSCAASGFTFSRNGMHWVRQAPGKGLEWVAVIANDGGNNYYADSVKGRFTISRDNSKNTLYLQMNSLRAEDTAVYYCAKAGYYYDSGTYYSQYYFDYWGQGTLVTVSS

SEQ ID NO: 6 - protein, 125 aa, Artificial Sequence
  Description of Artificial Sequence Synthetic polypeptide
QVQLQESGSGLVKPSQTLSLTCAVSGGSISSGDYSWSWIRQPPGKGLEWIAYIYHNGSAYYNPSLKSPVTISIDGSKNQFSLKLSSVTAADTAVYYCARMSNYDTSGYYDAFDIWGQGTMVTVSS

SEQ ID NO: 7 - protein, 120 aa, Artificial Sequence; identical to SEQ ID NO: 4
  Description of Artificial Sequence Synthetic polypeptide

SEQ ID NO: 8 - protein, 127 aa, Artificial Sequence
  Description of Artificial Sequence Synthetic polypeptide
EVQLVESGGGVVQPGRSLRLSCAASGFTFTSYGMHWVRQAPGKGLEWVAVISNDGSNIYYADSVEGRFTISRDNFKNTVYLQMNSLGAEDTAVYYCAKAGNYYDGSGYYSQYYFDNWGRGTLVTVSS

SEQ ID NO: 9 - protein, 120 aa, Artificial Sequence; identical to SEQ ID NO: 4
  Description of Artificial Sequence Synthetic polypeptide

SEQ ID NO: 10 - protein, 112 aa, Artificial Sequence
  Description of Artificial Sequence Synthetic polypeptide
DIVMTQSPDSLAVSLGERATINCKSSQSILYSSNNKNYLAWYQQKPGQPPKLLIYWASTRESGVPDRFSGSESGTDFTLTISSLQAEDVAVYYCQQYYSTPTFGPGTKVDIK

SEQ ID NO: 11 - protein, 107 aa, Artificial Sequence
  Description of Artificial Sequence Synthetic polypeptide
EIVLTQSPATLSLSPGERATLSCRASQNVSRYLAWYQQKPGQAPRLLIYDASNRATGIPARFSGSGSGTDFTLTISSLEPEDFAVYYCQQRRSWPPTFGGGTKVEIK

SEQ ID NO: 12 - protein, 112 aa, Artificial Sequence
  Description of Artificial Sequence Synthetic polypeptide
DIVMTQSPDSLAVSLGERATINCKSSQSVLYSSNNKSFLAWYQQKPGQPPSLLIHWASTRESGVPDRFSGSGSGTDFTLTISSLQAEDVAVYYCQQYYSTPTFGGGTKVEIK

SEQ ID NO: 13 - protein, 107 aa, Artificial Sequence; identical to SEQ ID NO: 11
  Description of Artificial Sequence Synthetic polypeptide

SEQ ID NO: 14 - protein, 107 aa, Artificial Sequence; identical to SEQ ID NO: 11
  Description of Artificial Sequence Synthetic polypeptide

SEQ ID NO: 15 - protein, 112 aa, Artificial Sequence
  Description of Artificial Sequence Synthetic polypeptide
DIVMTQSPDSLAVSLGERATINCKSSHSVLYDSNSKNYLAWYQQKPGQPPKLLIYWASTRDSGVPDRFSGSGSGTEFTLTISSLQAEDVALYYCQQYYSTPTFGGGTKVEIK

SEQ ID NO: 16 - protein, 107 aa, Artificial Sequence; identical to SEQ ID NO: 11
  Description of Artificial Sequence Synthetic polypeptide

SEQ ID NO: 17 - protein, 108 aa, Artificial Sequence
  Description of Artificial Sequence Synthetic polypeptide
SYELTQPPSVSVSPGQTARITCSGDALPKQYAYWYQQKPGQAPVLVIYKDTERPSGIPERFSGSSSGTTVTLTISGVQAEDEADYYCQSADSSDPYVVFGGGTKLTVL

SEQ ID NO: 18 - DNA, 432 bp, Artificial Sequence
  Description of Artificial Sequence Synthetic polynucleotide
gaggtkcagctggtggagtctgggggaggcgtggtccagcctgggaggtcccagagactctcctgtgcagcctctggattcactttcagaagatatgctatccactgggtccgccaggctccaggcaaggggctggagtgggtggcggttatctcaaatgatggaagcaataaattctacgcagagtccgtggagggccgattcaccgtctccagagacaattccatgaacacggtgtatctgcaaatgaacggcctgagagctgaggacacggctgtatattactgtgcgagagcgccggaatactgtagcggtggtagttgttacagtcaatactactttgactactggggccggggaaccctggtcaccgtctcctcggcctccaccaagggcccatcggtcttccccctggcaccctcctccaagagc

SEQ ID NO: 19 - DNA, 411 bp, Artificial Sequence
  Description of Artificial Sequence Synthetic polynucleotide
gaggtkcagctggtggagtctgggggaggcttggtccagcctggggggtccctgagactctcctgtgcagcctctggattcacatttagtgactattggatgagctgggtccgccaggctccagggaaggggctggagtgggtggccaacataaagtatgatggaagtgagaaatactatgtggactctgtgaagggccgattcaccacctccagagacaacgccaagaactcactgtatctgcaaatgaacagcctgagagccgaggacacggctgtgtattactgtgcaggattattatggttcggggagaaggcttttgatatctggggccaagggacaatggtcaccgtctcttcagcatccccgaccagccccaaggtcttcccgctgagcctcgacagcaccagc

SEQ ID NO: 20 - DNA, 432 bp, Artificial Sequence
  Description of Artificial Sequence Synthetic polynucleotide
gaggtkcagctggtggagtctgggggaggcgtggtccagcctgggaggtccctgagactctcctgtgcagcctctggattcaccttcagtaggaatggcatgcactgggtccgccaggctccaggcaaggggctggagtgggtggcagttatagcaaatgatggaggtaataactactatgcagactccgtgaagggccgattcaccatctccagagacaattccaagaacacgctgtatctgcaaatgaacagcctgagagctgaggacacggctgtgtattactgtgcgaaggctgggtattactatgatagtggtacttattactctcagtactactttgactactggggccagggaaccctggtcaccgtctcctcagcctccaccaagggcccatcggtcttccccctggcaccctcctccaagagc

SEQ ID NO: 21 - DNA, 426 bp, Artificial Sequence
  Description of Artificial Sequence Synthetic polynucleotide
caggtgcagctgcaggagtcgggctcaggactggtgaagccttcccagaccctgtccctcacctgcgctgtctctggtggctccatcagcagtggtgattactcctggagctggatccggcagccaccagggaagggcctggagtggattgcatacatctatcataatgggagcgcctactacaacccgtccctcaagagtccagtcaccatatcaatagacgggtccaagaaccagttctccctgaagctgagctctgtgaccgccgcggacacggccgtgtattactgtgccagaatgtcaaattatgatactagtggttattatgatgcttttgatatctggggccaagggacaatggtcaccgtctcttcagcctccaccaagggcccatcggtcttccccctggcaccctcctccaagagc

SEQ ID NO: 22 - DNA, 411 bp, Artificial Sequence; identical to SEQ ID NO: 19
  Description of Artificial Sequence Synthetic polynucleotide

SEQ ID NO: 23 - DNA, 432 bp, Artificial Sequence
  Description of Artificial Sequence Synthetic polynucleotide
gaggtkcagctggtggagtctgggggaggcgtggtccagcctgggaggtccctgagactctcctgtgcagcctctggattcaccttcactagctatggcatgcactgggtccgccaggctccaggcaaggggctggagtgggtggcagttatatcaaatgatggaagtaatatatactatgcagactccgtggagggccgattcaccatctccagagacaatttcaagaacacggtgtatctgcaaatgaacagcctgggggctgaggacacggctgtgtactattgtgcgaaggctggcaattactatgatggtagtggttactactctcagtactactttgacaactggggccggggaaccctggtcaccgtctcctcagcctccaccaagggcccatcggtcttccccctggcaccctcctccaagagc

SEQ ID NO: 24 - DNA, 411 bp, Artificial Sequence; identical to SEQ ID NO: 19
  Description of Artificial Sequence Synthetic polynucleotide

SEQ ID NO: 25 - DNA, 375 bp, Artificial Sequence
  Description of Artificial Sequence Synthetic polynucleotide
gacatcgtgatgacccagtctccagactccctggctgtgtctctgggcgagagggccaccatcaactgcaagtccagccagagtattttatacagctccaacaataagaactacttagcttggtaccagcagaaaccaggacagcctcctaaactgctcatttactgggcatctacccgggaatccggggtccctgaccgattcagtggcagcgagtctgggacagatttcactctcaccatcagcagcctgcaggctgaagatgtggcagtttattactgtcagcaatattatagtactcccactttcggccctgggaccaaagtggatatcaaacgaactgtggctgcaccatctgtcttcatcttcccaccc

SEQ ID NO: 26 - DNA, 952 bp, Artificial Sequence
  Description of Artificial Sequence Synthetic polynucleotide
gaaattgtgttgacrcagtctccagccaccctgtctttgtctccaggggaaagagccaccctctcctgcagggccagtcagaatgttagcagatacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgctgtttattactgtcagcagcgtaggagctggcctcccactttcggcggagggaccaaggtggagatcaaacgaactgtggctgcaccatctgtcttcatcatcccaccatctgatgagcagttgaaatctggaactgcctcagagcagatggagggaagatgaagacggatcgtgcacccacagttggattgatctccacatggggccagaaaatgtggggaggcagaggggagctcctgacggtaatgggacgcaattttacggggcgaggaaacttataatgcagagtgaacaaatgtacaaacccggcgataagcggggctggttatgccaatggggagaaggaagggacgggggaaaatagcacttggacacccctcgcggaaattcttataaaaaaactggctaaaatggcgcgcagggggggaaccgcgcccgccaaaaaatggggggcgctttatacccaaaaaaaaaaaaaaagggttgtttaaaaaaaacagatgtccaaatttttttctctcggggaaaaacaaaaaaaaaaaaaaatggtcgtacacccctccgccatctcatgaggtaagccgaacttcacaagagatgctgtccggtgcaactggacacactgcacgatacatacatccacacaaaaaacaaatactataacaataatatctatttagcatatattctcaatataaaa

SEQ ID NO: 27 - DNA, 375 bp, Artificial Sequence
  Description of Artificial Sequence Synthetic polynucleotide
gacatcgtgatgacccagtctccagactccctggctgtgtctctgggcgagagggccaccatcaactgcaagtccagccagagtgttttatacagctccaacaataagagcttcttagcttggtaccagcagaaaccaggacagcctcctagcctgctcattcactgggcatctacccgggaatccggggtccctgaccgattcagtggcagcgggtctgggacagatttcactctcaccatcagcagcctgcaggctgaagatgtggcagtttattactgtcagcaatattatagtactcccactttcggcggagggaccaaggtggagatcaaacgaactgtggctgcaccatctgtcttcatcttcccaccc

SEQ ID NO: 28 - DNA, 359 bp, Artificial Sequence
  Description of Artificial Sequence Synthetic polynucleotide
gaaattgtgttgacrcagtctccagccaccctgtctttgtctccaggggaaagagccaccctctcctgcagggccagtcagaatgttagcagatacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgctgtttattactgtcagcagcgtaggagctggcctcccactttcggcggagggaccaaggtggagatcaaacgaactgtggctgcaccatctgtcttcatcttcccacc

SEQ ID NO: 29 - DNA, 1000 bp, Artificial Sequence
  Description of Artificial Sequence Synthetic polynucleotide
gaaattgtgttgacrcagtctccagccaccctgtctttgtctccaggggaaagagccaccctctcctgcagggccagtcagaatgttagcagatacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgctgtttattactgtcagcagcgtaggagctggcctcccactttcggcggagggaccaaggtggagatcaaacgaactgtggctgcaccatctgtcacaatcttcccaaacgcggtcccaccaacaaaaccttgggcctccgccatagtctttcttcgcacgacaccccgcgaccaacactaggaaaaaggaggcccagacccaatcggccgaccgagggagagggagagagggggggcggggcgaggcaccaacttaaatagaaaagcaccaggggtcatgattaccttgttttttttaagggaaacgaggcaagcaaaaatataaatctaaggatcccagaccggaacctaaaatgttgctactcacgtggatcacccccagagtcgctcagctctagcggtgaaaaagaaaaacaaaaaaaaaaaatcgaaatcttccaagatatataaaaaaacaaaaagtggaagaatagggaatagatgaggaactaaataggaacttaaaaaaattaagagatgagagctacgggaggcccacccaaaaaaaaaaaaaaaaaagagtgaggataatacaaaaaaaataaggagaaaaagaataaaaaaaaacaaaaaagaaaaaatatatagtcataatacaaaaccatactccaaacaatttaaatattagaatccattaccctcactattacatcataaaaacacgccaccccacgagaaaaaaataaagagaatatcgtccaaacattctc

SEQ ID NO: 30 - DNA, 1000 bp, Artificial Sequence
  Description of Artificial Sequence Synthetic polynucleotide
gacatcgtgatgacccagtctccagactccctggctgtgtctctgggcgagagggccaccatcaactgcaagtccagccacagtgttttatacgactccaacagtaagaactacttagcttggtaccagcagaaaccaggacagcctcctaagctgctcatttactgggcatctacccgggattccggggtccctgaccgcttcagtggcagcgggtctgggacagagttcactctcaccatcagcagcctgcaggctgaagatgtggcactttattactgtcagcaatattacagtactcccactttcggcggagggaccaaggtggagatcaaacgaactgtggctgcaccatctgtcttcatcttcccaaggtcgaaccagattccccctagggaccacttcgacgagtacgcttggcggagaaagatagacaaaaaaatagggtacaaagtatggagacactagataattgtgctgggaaggggggggggggggggttctaatcggtaacgaacacggtccagcaaattgctaagaccgcagaaatagtaggggtacggggagaggaggtgtaagatgaaggataatcatatagaaaacagataattcggggaattaagagtcttaaaagataacgaaaacagaccggaaaacaaagaccaggagaggagagaaaaaaaaccctggggaataccaccgacagatgaaaaataatagcgaagaaatgcgtagggcggaaaaaggcgaaataaatgcgggaagaatggggaaatgtagtataacaaaccaggaaaggcggagggggaaacccccataatgaggtaaatataggagaggtgagagagagacaatactggtactaagggagaagaaaatctgacataagaaaagaatgaggagaaaaaaaaggccatctaaacaaaaaaaatacaaagagaaaaacagaaacaagatattcgggaaataagaaaagcaaaaagaacaaaagaaaaagata

SEQ ID NO: 31 - DNA, 1000 bp, Artificial Sequence
  Description of Artificial Sequence Synthetic polynucleotide
gaaattgtgttgacrcagtctccagccaccctgtctttgtctccaggggaaagagccaccctctcctgcagggccagtcagaatgttagcagatacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgctgtttattactgtcagcagcgtaggagctggcctcccactttcggcggagggaccaaggtggagatcaaacgaactgtggctgcaccatctgtcacccttcttcccaaacccgcacaaagatcctcttccccagtttctctaagaggaaaagaaacacccaaaggccccaaaagtggaaccttcctgaaccaaagagcgggcggggcggcctctactttgtcaccaatggcattttttggttgtgattgggttggcagggacattccccccccggcccggccagacaacacttgagttttacccccagactaaaagggttcttgtgaagcaaaaagacgataaaccaatagttctctccaacttcccaaggcaaaagggcttatggagaaaacggcccttaaacctttccaaacccgaggaggcacccactaaaaaaccccagccggagccccacccacggccccaggggcaaaaaccctaagaaaatgggtgtctgagacccccctctattcccctttgggaaaagataccatatattgtttttccaggaccaccatcccacaacatttttttttaacaaacccacccgcaccccccactaagttggagccctcaacaaagaaaagaagcccccaattgatttgcacatactcactatatacaagttaaaaaggggaggggtcaaacactgtggaccggggcgcccgtcaaaactaacaggtcacccccccaacctccgaaaaaaaaa

SEQ ID NO: 32 - protein, 30 aa, Artificial Sequence
  Description of Artificial Sequence Synthetic polypeptide
KYALADASLKMADPNRFRGKDLPVLDQLLE

SEQ ID NO: 33 - protein, 527 aa, Artificial Sequence
  Description of Artificial Sequence Synthetic polypeptide
MGGAAARLGAVILFVVIVGLHGVRGKYALADASLKMADPNRFRGKDLPVLDQLLEVPVWKEADTTLFCASDAKAHETEVHNVWATHACVPTDPNPQEIDLENVTENFNMWKNNMVEQMQEDVISLWDQSLKPCVKLTPPCVTLHCTNANLTKANLTNVNNRTNVSNIIGNITDEVRNCSFNMTTELRDKKQKVHALFYKLDIVPIEDNNDSSEYRLINCNTSVIKQPCPKISFDPIPIHYCTPAGYAILKCNDKNFNGTGPCKNVSSVQCTHGIKPVVSTQLLLNGSLAEEEIIIRSENLTNNAKTIIVHLNKSVVINCTRPSNNTRTSITIGPGQVFYRTGDIIGDIRKAYCEINGTEWNKALKQVTEKLKEHFNNKPIIFQPPSGGDLEITMHHFNCRGEFFYCNTTRLFNNTCIANGTIEGCNGNITLPCKIKQIINMWQGAGQAMYAPPISGTINCVSNITGILLTRDGGATNNTNNETFRPGGGNIKDNWRNELYKYKVVQIEPLGVAPTRAKRRVVEREKR

SEQ ID NO: 34 - protein, 15 aa, Human immunodeficiency virus
SINIGPGQVFYRTDG

SEQ ID NO: 35 - DNA, 1431 bp, Artificial Sequence
  Description of Artificial Sequence Synthetic polynucleotide
gctagcaccatggagacagacacactcctgctatgggtactgctgctctgggttccaggttccactggtgacgaggtgcagctggtggagtctgggggaggcttggtccagcctggggggtccctgagactctcctgtgcagcctctggattcacatttagtgactattggatgagctgggtccgccaggctccagggaaggggctggagtgggtggccaacataaagtatgatggaagtgagaaatactatgtggactctgtgaagggccgattcaccacctccagagacaacgccaagaactcactgtatctgcaaatgaacagcctgagagccgaggacacggctgtgtattactgtgcaggattattatggttcggggagaaggcttttgatatctggggccaagggacaatggtcaccgtctcttcagcatccccgaccagccccaaggtcttcccgctgagcctcgacagcaccagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggtacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacgccacgtaccgtgtggtcagcgtcctcaccgtcctgcaccaggactggctgaatggcaaggagtacaagtgcaaggtctccaacaaagccctcccagcccccatcgcggcaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgaggatcc

SEQ ID NO: 36 - protein, 471 aa, Artificial Sequence
  Description of Artificial Sequence Synthetic polypeptide
METDTLLLWVLLLWVPGSTGDEVQLVESGGGLVQPGGSLRLSCAASGFTFSDYWMSWVRQAPGKGLEWVANIKYDGSEKYYVDSVKGRFTTSRDNAKNSLYLQMNSLRAEDTAVYYCAGLLWFGEKAFDIWGQGTMVTVSSASPTSPKVFPLSLDSTSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 729 bp, Artificial Sequence
  Description of Artificial Sequence Synthetic polynucleotide
gctagcaccatggagacagacacactcctgctatgggtactgctgctctgggttccaggttccactggtgacgaaattgtgttgacacagtctccagccaccctgtctttgtctccaggggaaagagccaccctctcctgcagggccagtcagaatgttagcagatacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgctgtttattactgtcagcagcgtaggagctggcctcccactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgatctagagggccc

SEQ ID NO: 38 - protein, 235 aa, Artificial Sequence
  Description of Artificial Sequence Synthetic polypeptide
METDTLLLWVLLLWVPGSTGDEIVLTQSPATLSLSPGERATLSCRASQNVSRYLAWYQQKPGQAPRLLIYDASNRATGIPARFSGSGSGTDFTLTISSLEPEDFAVYYCQQRRSWPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - DNA, 1514 bp, Artificial Sequence
  Description of Artificial Sequence Synthetic polynucleotide
gctagcaccatggagacagacacactcctgctatgggtactgctgctctgggttccaggttccactggtgacgaggttcagctggtggagtctgggggaggcgtggtccagcctgggaggtccctgagactctcctgtgcagcctctggattcaccttcactagctatggcatgcactgggtccgccaggctccaggcaaggggctggagtgggtggcagttatatcaaatgatggaagtaatatatactatgcagactccgtggagggccgattcaccatctccagagacaatttcaagaacacggtgtatctgcaaatgaacagcctgggggctgaggacacggctgtgtactattgtgcgaaggctggcaattactatgatggtagtggttactactctcagtactactttgacaactggggccggggaaccctggtcaccgtctcctcagcctccaccaagggcccatcggtcttccccctggcaccctcctccaagagcacctctgggggcacagcggccccgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggtacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacgccacgtaccgtgtggtcagcgtcctcaccgtcctgcaccaggactggctgaatggcaaggagtacaagtgcaaggtctccaacaaagccctcccagcccccatcgcggcaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgaggatccactagtccagtgtggtggaattctgcagatatccagcacagtggcggccgctcgagtctaga

SEQ ID NO: 40 - protein, 478 aa, Artificial Sequence
  Description of Artificial Sequence Synthetic polypeptide
METDTLLLWVLLLWVPGSTGDEVQLVESGGGVVQPGRSLRLSCAASGFTFTSYGMHWVRQAPGKGLEWVAVISNDGSNIYYADSVEGRFTISRDNFKNTVYLQMNSLGAEDTAVYYCAKAGNYYDGSGYYSQYYFDNWGRGTLVTVSSASTKGPSVFPLAPSSKSTSGGTAAPGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - DNA, 744 bp, Artificial Sequence
  Description of Artificial Sequence Synthetic polynucleotide
gctagcaccatggagacagacacactcctgctatgggtactgctgctctgggttccaggttccactggtgacgacatcgtgatgacccagtctccagactccctggctgtgtctctgggcgagagggccaccgtcaactgcaagtccagccacagtgttttatacgactccaacagtaagaactacttagcttggtaccagcagaaaccaggacagcctcctaagctgctcatttactgggcatctacccgggattccggggtccctgaccgcttcagtggcagcgggtctgggacagagttcactctcaccatcagcagcctgcaggctgaagatgtggcactttattactgtcagcaatattacagtactcccactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgatctagagggccc

SEQ ID NO: 42 - protein, 240 aa, Artificial Sequence
  Description of Artificial Sequence Synthetic polypeptide
METDTLLLWVLLLWVPGSTGDDIVMTQSPDSLAVSLGERATVNCKSSHSVLYDSNSKNYLAWYQQKPGQPPKLLIYWASTRDSGVPDRFSGSGSGTEFTLTISSLQAEDVALYYCQQYYSTPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2014/0302062 A1
(43) Pub. Date: Oct. 9, 2014

(54) HERPES SIMPLEX VIRUS

(71) Applicants: Barton F. Haynes, Durham, NC (US); Hua-Xin Liao, Durham, NC (US); M. Anthony Moody, Durham, NC (US); Georgia D. Tomaras, Durham, NC (US); Jerome Kim, Fort Detrick, MD (US); Nelson Michael, Fort Detrick, MD (US)

(72) Inventors: Barton F. Haynes, Durham, NC (US); Hua-Xin Liao, Durham, NC (US); M. Anthony Moody, Durham, NC (US); Georgia D. Tomaras, Durham, NC (US); Jerome Kim, Fort Detrick, MD (US); Nelson Michael, Fort Detrick, MD (US)

(73) Assignees: U.S. ARMY MEDICAL RESEARCH AND MATERIEL COMMAND, Fort Detrick, MD (US); DUKE UNIVERSITY, Durham, NC (US)

(21) Appl. No.: 14/110,537

(22) PCT Filed: Apr. 9, 2012
(86) PCT No.: PCT/US12/32743

§ 371 Date: May 22, 2014

Related U.S. Application Data
(60) Provisional application No. 61/473,543, filed on Apr. 8, 2011.

(51) Int. Cl. C07K 16/08

(52) U.S. Cl. CPC C07K 16/087
USPC 424/159.1; 530/389.4; 536/23.53; 435/320.1

(57) ABSTRACT

The present invention relates, in general to herpes simplex virus (HSV) and, particular, to antibodies that are specific for glycoprotein D (gD) of HSV. The invention also relates to prophylactic and therapeutic uses of such antibodies.

This application claims priority from U.S. Provisional Application No. 61/473,543, filed Apr. 8, 2011, the entire content of which is incorporated herein by reference.

This invention was made with government support under Grant No. CHAVI U19 AI067854 awarded by the National Institutes of Health. The government has certain rights in the invention.

### TECHNICAL FIELD

The present invention relates, in general to herpes simplex virus (HSV) and, in particular, to antibodies that are specific for glycoprotein D (gD) of HSV. The invention also relates to prophylactic and therapeutic uses of such antibodies.

### BACKGROUND

HSV types 1 and 2 are enveloped DNA viruses of the herpesvirus family that are common causes of human disease. HSV-1 is frequently acquired early in life such that ˜50% of 5-year-old children in the US have evidence of infection. Acquisition continues throughout life and 70-90% of the elderly have evidence of prior infection. HSV-2 acquisition is more sporadic with infection rates increasing throughout adolescence and data shows that ˜20% of US adults have evidence of infection, although, in certain populations, the rates can be substantially higher, in some cases up to 80%.

Herpesvirus infections are acquired through person-to-person contact and the site of entry is skin and/or mucous membranes. The viruses bind to cellular receptors via proteins expressed on the surface of virions, including gD, and interaction of these virus receptors with host receptors triggers the events of virus fusion and host cell infection. Once infection is established in the host, the virus can infect multiple cell types and can cause disease ranging from localized blistering (vesicles), such as is seen in a cold sore, local spread of vesicular rash, dissemination of the vesicular rash, invasion of the bloodstream, infection of internal organs (including the liver), and infection of the central nervous system (including the brain). More extensive disease is associated with increasing degrees of morbidity and mortality.

Once infection has occurred, all herpesvirus infections establish latency in the host. HSV-1 and HSV-2 infect nerve cells, typically peripheral ganglia, and can remain dormant for days to years. Reactivation occurs following signaling events that are poorly understood. Once reactivation occurs, the virus replicates and either asymptomatic shedding of the virus or shedding in the context of disease manifestations can occur. It is these periods of virus replication that are associated with the common manifestations of recurrent HSV disease, including cold sores around the mouth and outbreaks of genital herpes. During periods of such outbreaks, transmissible virus is shed and while symptomatic outbreaks are associated with higher levels of virus shedding, asymptomatic shedding is known to occur frequently. Studies of adult women infected with genital HSV-2 suggest that there is a 1 in 100 chance on any day of asymptomatic shedding of infectious virus.

While many infections with herpes viruses are asymptomatic in healthy hosts or only cause relatively mild or localized disease, infection in hosts with compromised immune systems can be devastating. In particular, populations at very high risk for disseminated or central nervous system disease include newborn infants, patients with inborn errors of the immune system, patients with acquired immune deficiencies (e.g., HIV infection), patients undergoing chemotherapy for malignancies, and the elderly. Such patients are at risk of more severe primary disease, more severe recurrent disease, difficulty controlling infection once established, shorter periods of latency compared to healthy hosts, increased rates of asymptomatic shedding, and a higher likelihood of dissemination.

The immune response to HSV involves innate and adaptive immunity. As with all viral infections, both cell-mediated and humoral responses are critical. The critical importance of humoral immunity has been suggested by studies of HSV transmission around the time of birth (i.e., perinatal or congenital HSV) where infants born to women experiencing primary HSV disease are more likely to acquire HSV than infants born to women with recurrent HSV. This is thought to be due to transplacental transfer to the infant of IgG antibodies produced by the mother that provide a degree of protection. For this reason, an effective vaccine that can induce such antibodies and/or human mAbs that can be passively administered could provide protection to infants against this disease.

To date, efforts at producing an effective vaccine against HSV have proven disappointing and no approved, commercially available vaccine exists. Thus, options for the control of HSV infection in vulnerable or infected populations have focused on drug therapies. A number of drugs are available and most target the DNA replication machinery of the virus. In particular, drugs that target virally encoded thymidine kinase, such as acyclovir, have proven highly effective. As with all antimicrobial therapies, however, resistance occurs and often it occurs in the most vulnerable hosts. When resistance develops, alternative drugs with less desirable side effect profiles may be used, however, alternative preventative and therapeutic strategies are needed.

Humanized monoclonal antibody therapeutics have become commonplace and represent a growing market. Such antibodies can exhibit persistence in patients similar to endogenously produced antibodies and have the advantage of high specificity for their targets. An antibody targeted against respiratory syncytial virus (RSV), palivizumab (Synagis®), has proven effective in preventing severe RSV disease in vulnerable infants.

Humanized antibodies are typically derived from non-human animal models and are engineered to give them characteristics of human antibodies. This engineering is designed to prevent rapid clearance through production of immune complexes and also to prevent the development of immune response against the foreign protein. Antibodies derived from humans directly do not require such engineering steps as the antibodies will not be recognized as foreign by most or all human subjects.

The present invention relates, at least in part, to anti-HSV gD antibodies derived from a vaccinated human subject and rescued using recombinant DNA techniques. The invention further relates to the use of such anti-HSV gD antibodies in passive immunotherapy regimens.

### SUMMARY OF THE INVENTION

In general, the invention relates to anti-HSV antibodies. More particularly, the invention relates to antibodies specific for gD of HSV. The invention further relates to methods of using such antibodies both prophylactically and therapeutically.

Objects and advantages of the present invention will be clear from the description that follows.

### BRIEF DESCRIPTION OF THE DRAWINGS

FIG. 1. Memory B cells from RV135 subject T141442 stained with HSV gD antigen-specific reagents.

FIGS. 2A and 2B. (FIG. 2A) Heavy and light chain amino acid sequences of seven human antibodies specific for gD, with CDRs noted. (FIG. 2B) Heavy and light chain gene sequences that include sequences encoding the amino acid sequences shown in FIG. 2A. (mAb 5157-H005157 and K003927; mAb 5158-H005158 and K003928; mAb 5159-H005159 and K003929; mAb 5160-H005160, K003930 and L001844; mAb 5188-H005188 and K003946; mAb 5190-H005190 and K003948; and mAb 5192-H005192 and K003949.)

FIGS. 3A-3C. Mapping of mAbs. (FIG. 3A) Monoclonal antibody Ab5157. (FIG. 3B). Monoclonal antibody Ab5190. (FIG. 3C) Monoclonal antibody Ab5188.

FIGS. 4A-4C. (FIG. 4A) Herpes simplex gD bound to human receptor HveA (FIG. 4B) Same views as shown in FIG. 4A with residues shown in FIGS. 3A and 3B to be critical for binding of mAbs 5157 (CH41) and 5190 (CH43) highlighted in yellow and pointed at by arrows. (FIG. 4C) Same views of the crystal structure shown in FIG. 4A with the amino acids shown in FIG. 3C to be critical for binding for mAb 5188 (CH42) highlighted in yellow and pointed at by an arrow.

FIG. 5. RV144/135 sorted antibodies.

FIG. 6. Two gD monoclonal antibodies. CH42HCAAA has a unique amino acid sequence (underlined at the start of the constant region). The constant region sequence of CH42 is IgA2-IgG1_AAA chimeric-the original CH42 heavy chain was IgA2.

### DETAILED DESCRIPTION OF THE INVENTION

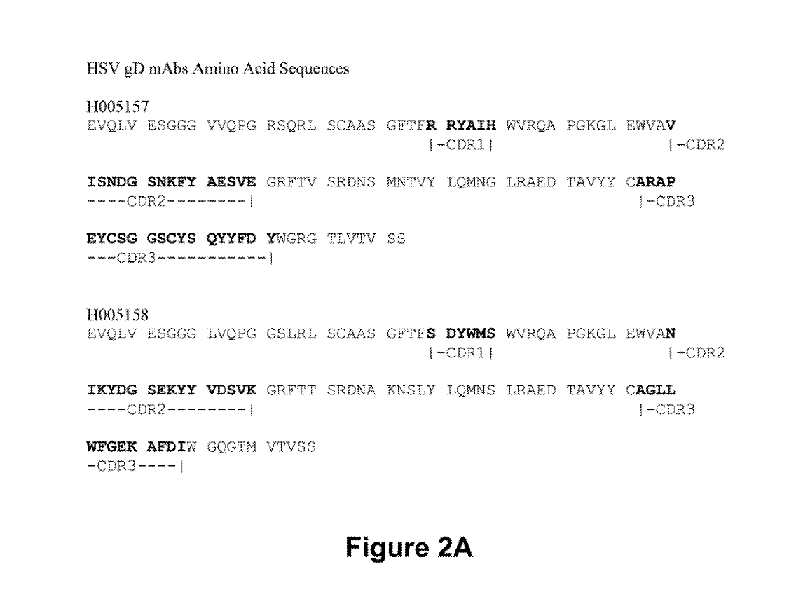

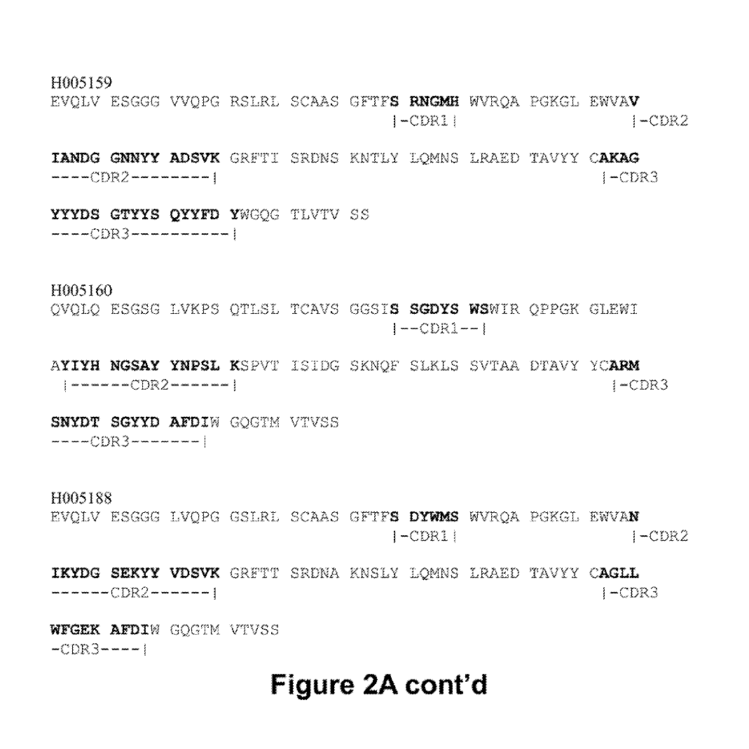

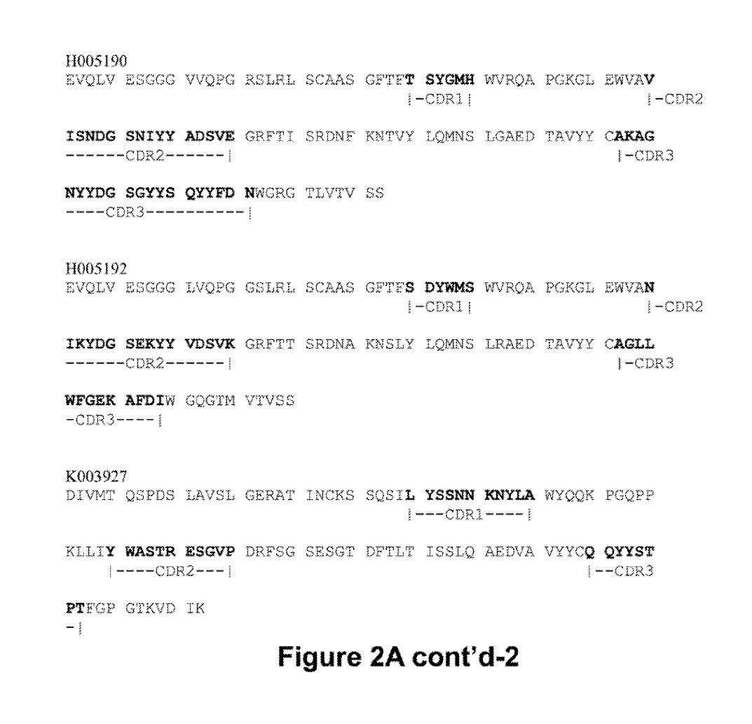

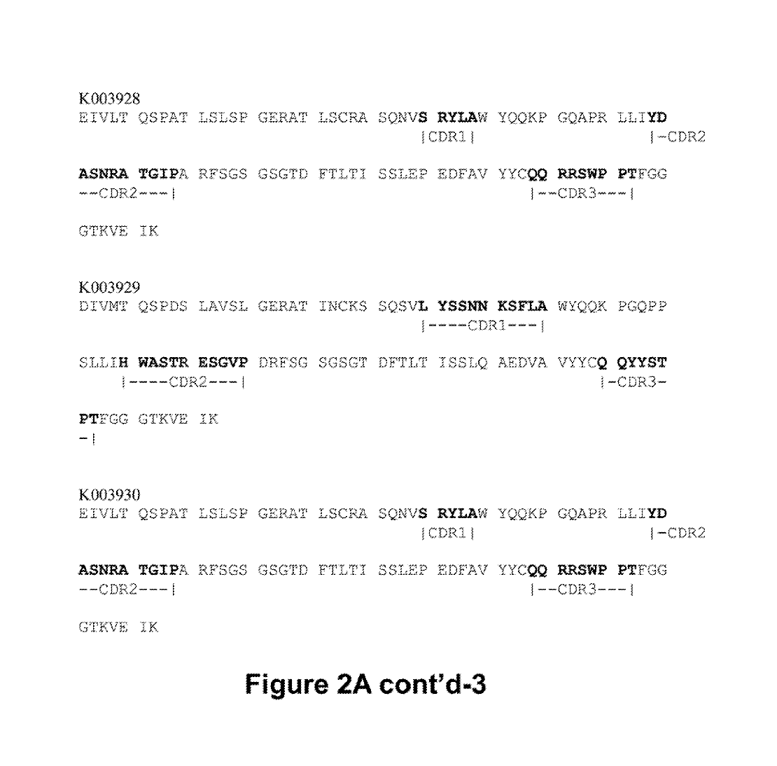

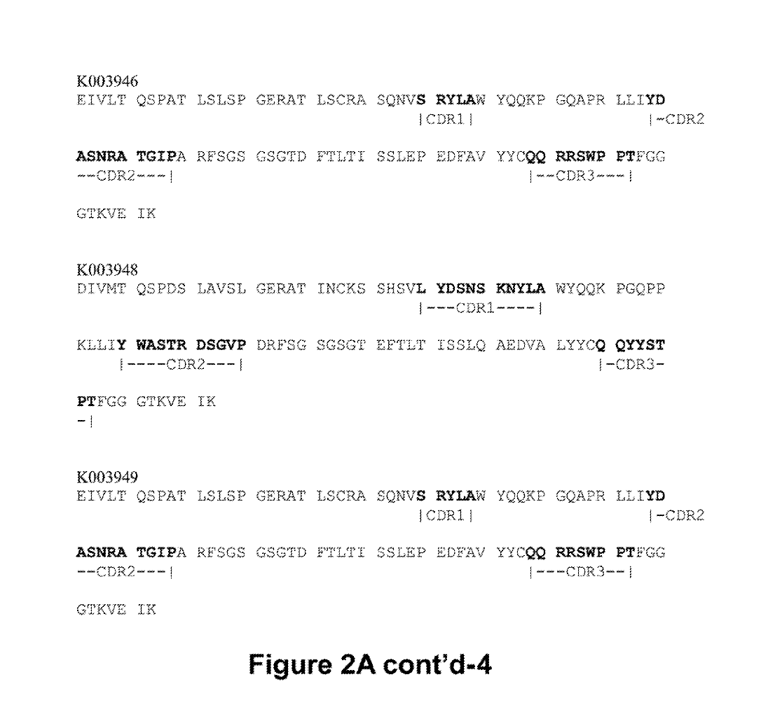

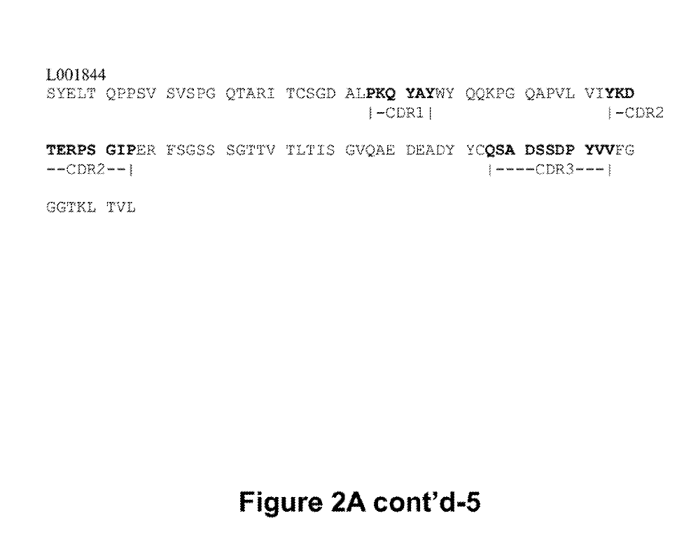

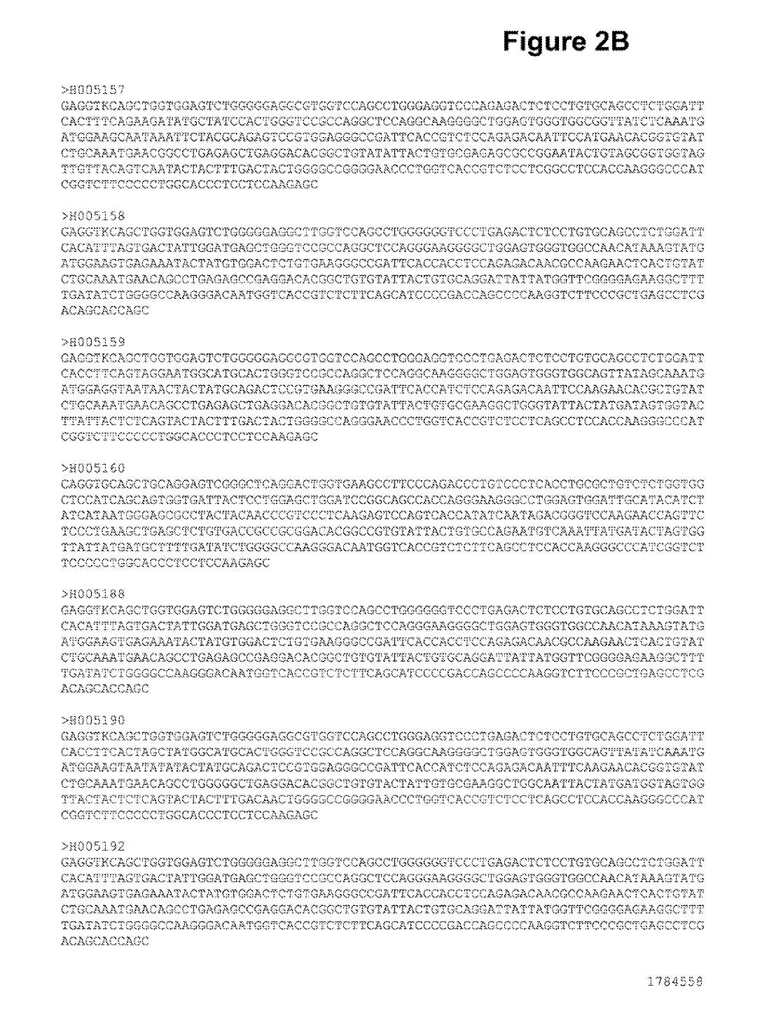

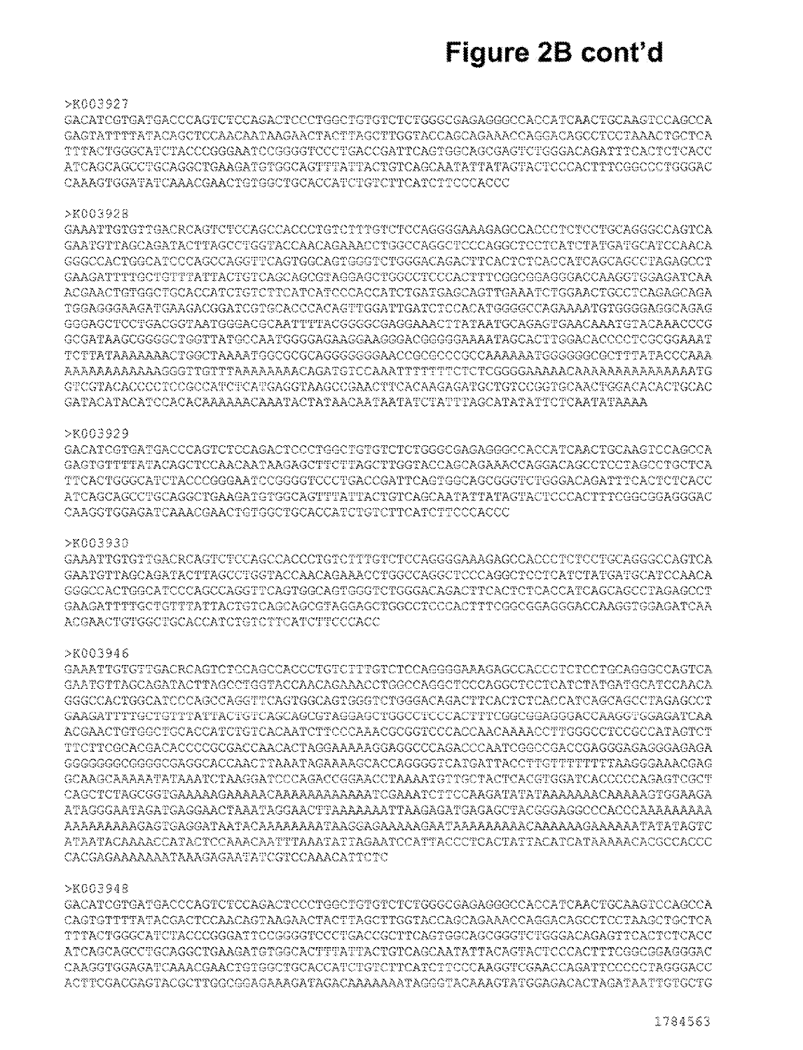

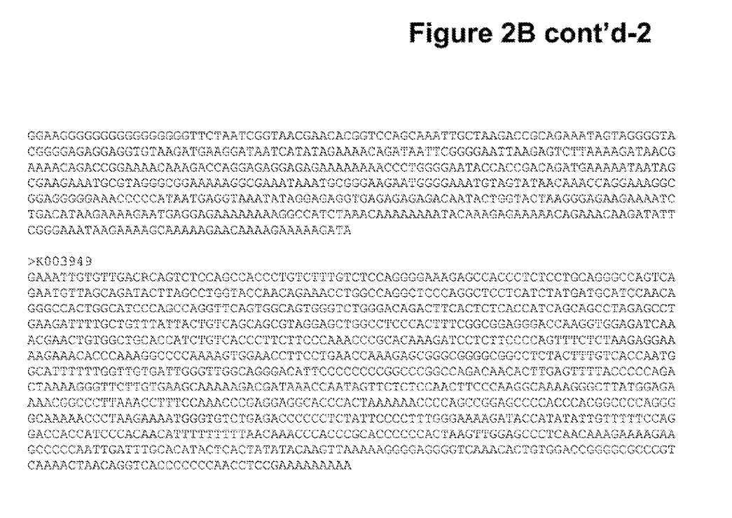

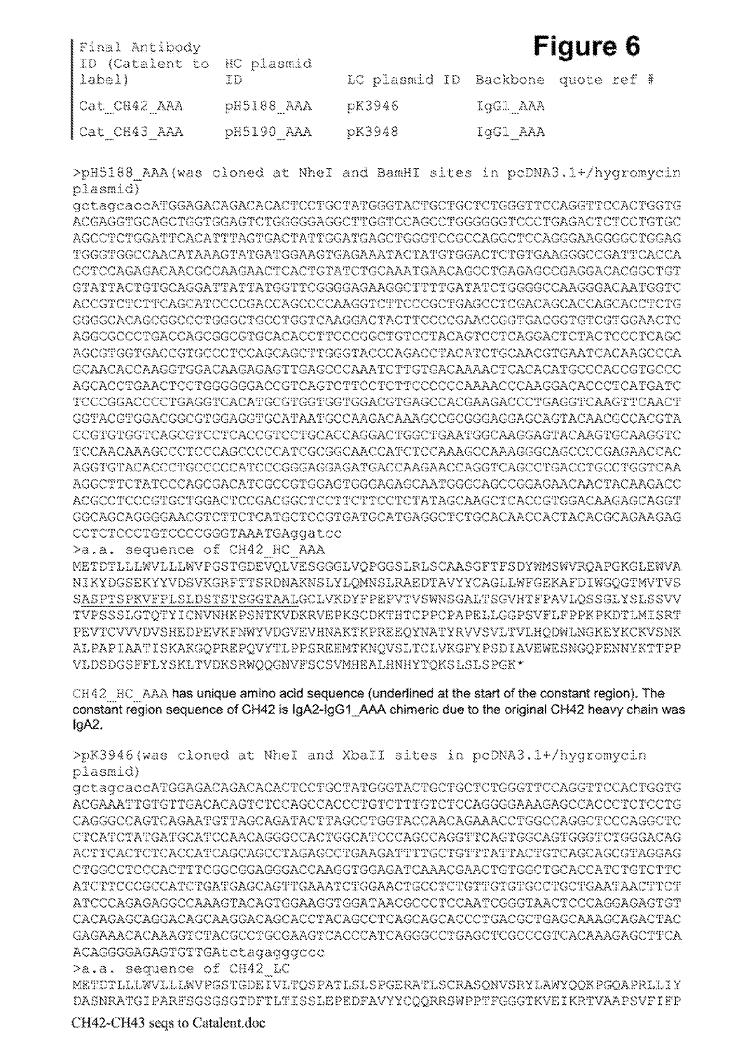

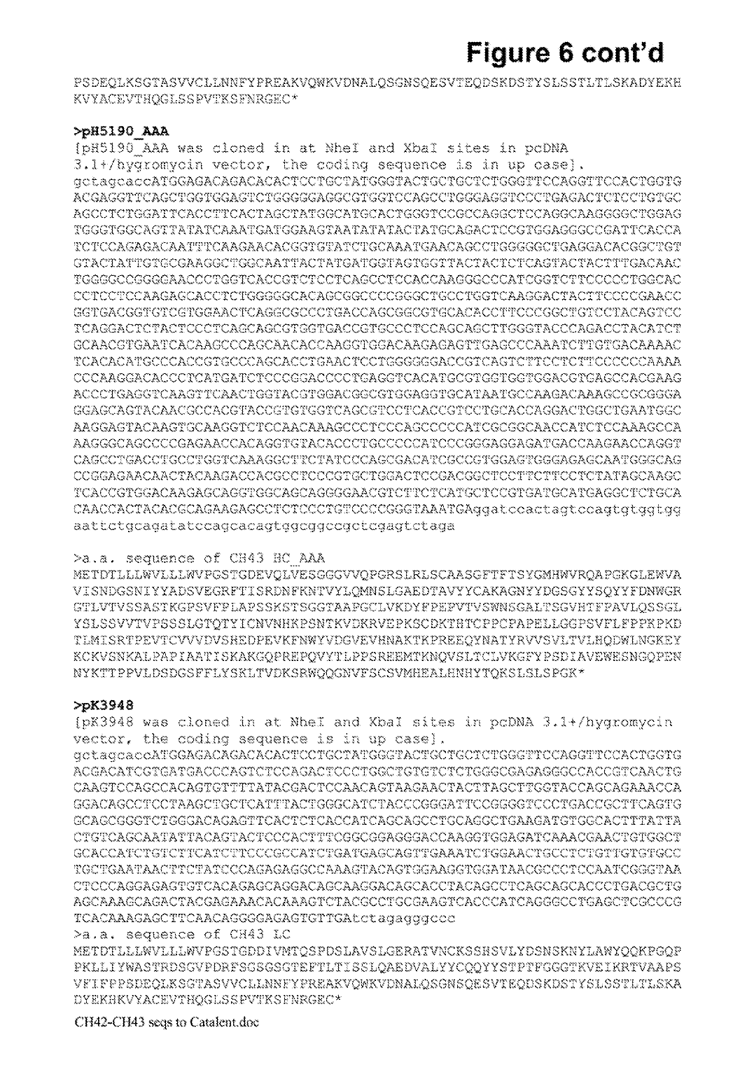

The present invention results, at least in part, from the identification of human antibodies specific for glycoprotein D (gD) of HSV (see Examples below). FIG. 2A includes heavy and light chain amino acid sequences of seven human antibodies specific for gD (with CDRs noted). FIG. 2B includes heavy and light chain gene sequences that include sequences encoding the amino acid sequences shown in FIG. 2A. FIG. 6 includes heavy and light chain amino acid sequences of two gD monoclonal antibodies and nucleic acid sequences encoding same. The invention relates to antibodies specific for gD of HSV, for example, antibodies that comprise a heavy and/or light chain as set forth in FIG. 2A or FIG. 6, or at least one or more CDR's of such chains. The invention also includes antibodies having the binding specificity of mAb 5157, 5158, 5159; 5160; 5188, 5190, 5192 or the antibodies set forth in FIG. 6. The invention further includes nucleic acid sequences encoding such amino acid sequences/antibodies. The invention also relates to prophylactic and therapeutic uses of such antibodies.

Antibodies specific for gD that are suitable for use in the prophylactic/therapeutic methods of the invention include dimeric, trimeric and multimeric antibodies, bispecific antibodies, chimeric antibodies, human and humanized antibodies, recombinant and engineered antibodies, and antigen-binding fragments thereof (e.g., Fab′, F(ab′)₂ fragments). Also suitable are single domain antibodies, Fv, single chain Fv, linear antibodies, diabodies, etc. The techniques for preparing and using various antibody-based constructs and fragments are well known in the art (see, for example, Kohler and Milstein, Nature 256:495 (1975), Kosbor et al, Immunol. Today 4:72 (1983), Cote et al, PNAS 80:2026 (1983), Morrison et al , PNAS 81:6851 (1984), Neuberger et al, Nature 312:604 (1984), Takeda et al, Nature 314:452 (1985), USP 4,946,778, EP 404,097, WO93/11161, Zapata et al, Prot. Eng. 8:1057 (1995) and Liao et al, J. Virol. Methods 158(1-2):171-179 (2009)).

Antibodies of the invention can be expressed in a system that produces them as IgG1 antibodies, the dominant type present in human plasma (Liao et al, J. Virol. Methods 158(1-2):171-179 (2009) and Smith et al, Nature Protocols 4(3)(January 1):372-384 (2009)). IgG1 antibodies can be passed through the placenta to infants prior to birth and can also become available at mucosal surfaces active or passive transport. In addition to the IgG1 expression system, antibodies of the invention can be expressed as other isotypes, in particular, as an IgA1 or IgA2 antibody (Carayannopoulos et al, Proc. Natl. Sci. USA 91(8) (August 30):8348-8352 (1994)). Such antibodies can provide additional protection at mucosal surfaces.

The antibodies of the invention can be used, for example, in humans, in a variety of prophylactic/therapeutic regimens. The antibodies can be used in passive immunotherapy strategies to prevent or treat HSV disease during pregnancy. The antibodies can also be used to prevent or treat perinatally acquired/congenital HSV in infants. The antibodies can be used to treat infection with drug-resistant HSV in immunocompromised or immunocompentent hosts.

Antibodies of the invention can be used prophylactically and/or therapeutically in mmunocompromised as well as immunocompetent hosts, including in subjects (e.g., humans) suffering from primary or secondary immunodeficiency and in subjects (e.g., humans) undergoing cancer chemotherapy or bone marrow transplantation. Antibodies of the invention also find use as adjunctive therapeutics in combination with other anti-HSV therapies.

The antibodies, or antibody fragments, of the invention can be formulated using standard techniques. Advantageously, the antibody/fragment is present in a composition, for example, a sterile composition suitable for injection (e.g., intramuscularly) or intravenous infusion. The composition can also take the form of a cream or ointment suitable for administration to skin or a mucosal surface (e.g., in the context of a microbicide for the prevention of HSV infection in a susceptible population). The composition can also be present as a formulation suitable administration to the eye for the prevention or treatment of HSV disease of the eye (including corneal disease, conjunctival disease, and surrounding structures). The optimum amount and route of administration can vary with the antibody/fragment, the patient and the effect sought. Optimum dosing strategies can be readily established by one skilled in the art.

Certain aspects of the invention are described in greater detail in the non-limiting Examples that follow (see also PCT/US07/07399, filed Mar. 26, 2007, U.S. application Ser. No. 12/225,541, filed Sep. 24, 2008, PCT/US2010/002770, filed Oct. 18, 2010, U.S. Provisional Application No. 61/407,299, filed Oct. 27, 2010 and Rerks-Ngarm et al, NEJM 361:2209-30 (2009)). Also incorporated by reference is a U.S. Provisional Application filed Apr. 8, 2011, entitled “Herpes Simplex Virus Vaccine”, Attorney Docket 01579-1688.

### EXAMPLE 1

### Isolation of Antibodies from a Subject Immunized in RV135 Study (AVLAC-prime gp120-boost)

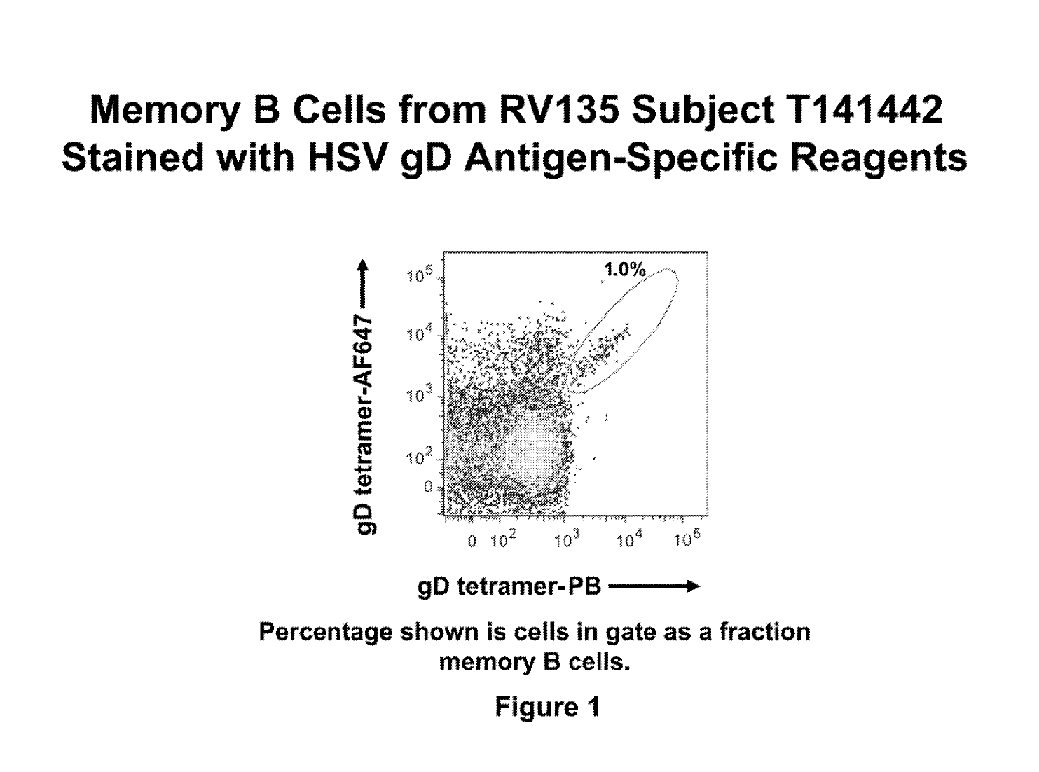

Flow cytometry data showing the population sorted to obtain HSV gD mAbs is provided in FIG. 1. Cells shown in the gate are memory B cells (live CD3/14/16/235a⁻ CD19⁺ surface IgD⁻) stained with B cell tetramer specific for the HSV gD sequence. Of memory B cells, 1.0% were labeled using this technique (dual color antigen-specific staining) and were sorted as individual cells into 96-well plates. Using recombinant DNA techniques, human mAbs were created from these cells (Liao et al, J. Virol. Methods 158(1-2):171-179 (2009) and Smith et al, Nature Protocols 4(3)(January 1):372-384 (2009)). Of nine heavy chains isolated from this sort, seven were specific for the gD sequence when assayed (see the heavy and light chain gene sequences set forth in FIG. 2). mAbs 5157, 5159, 5160 and 5190 are IgG1 antibodies and mAbs 5158, 5188 and 5192 are IgA2 antibodies.

The tetramer used to stain and sort in this experiment was based on the following sequence:

biotin-
KKKKYALADASLKMADPNRFRGKDLPVLDQLLE

This tetramer was prepared using standard techniques (see, for example, application Ser. No. 12/320,709).

### EXAMPLE 2

### Mapping of Isolated mAbs to Alanine-Substituted gD Peptides

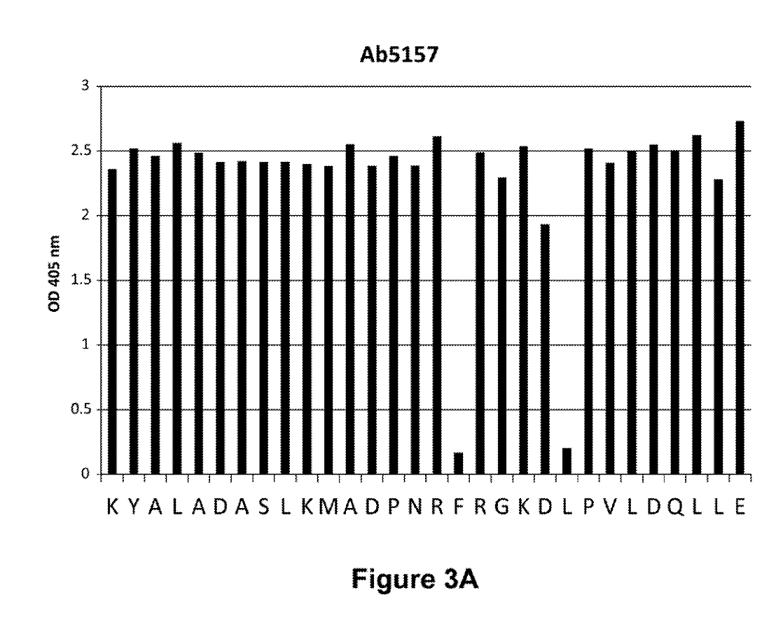

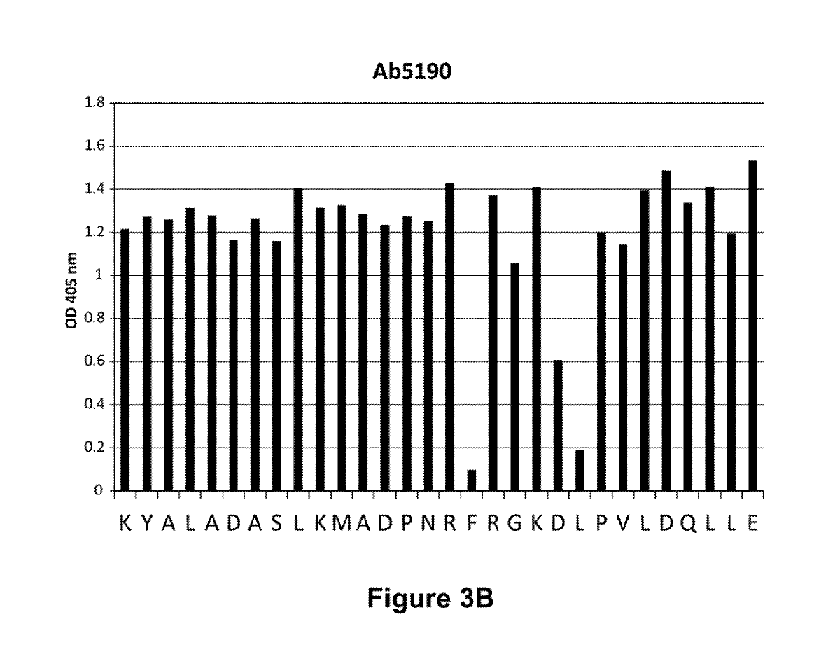

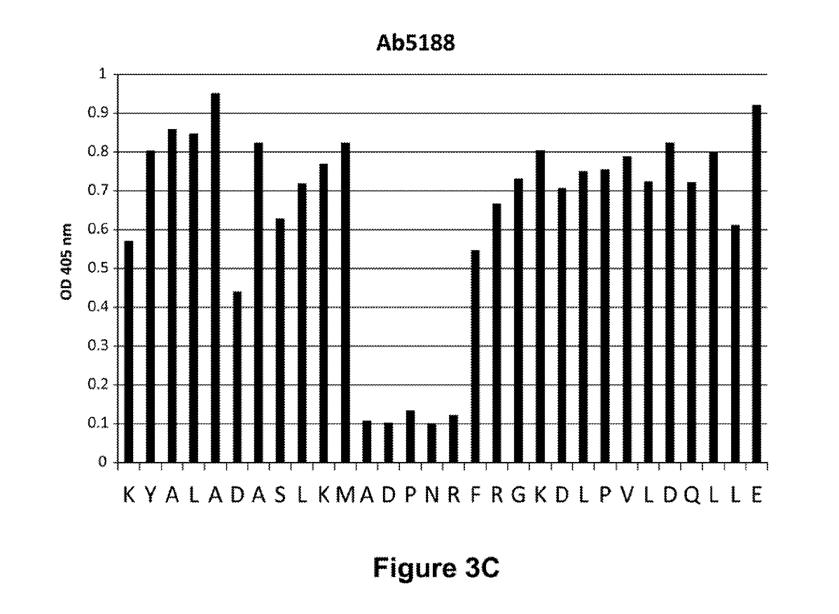

ELISA data of mapping of the residues critical for mAb binding for mAb 5157 (CH41) are shown in FIG. 3A. Assay results are nearly equivalent for all amino acid substitutions except for the phenylalanine (F) at position 17 and the leucine (L) at position 22 that show dramatic reductions in binding. In addition, a slight reduction is seen for substitution at position 21 (aspartic acid, D).

ELISA data of mapping of the residues critical for mAb binding for mAb 5190 (CH43) are shown in FIG. 3B. Similar to the results for CH41, the assay results are nearly equivalent for all amino acid substitutions except for the phenylalanine (F) at position 17 and the leucine (L) at position 22 that show dramatic reductions in binding. A smaller reduction is seen for substitution at position 21 (aspartic acid, D).

ELISA data of mapping of the residues critical for mAb binding for mAb 5188 (CH42) are shown in FIG. 3C. Assay results show that amino acid substitutions at positions 12-16 (ADPNR=alanine−aspartic acid−proline−asparagine=arginine) reduce binding to near background. Substitution of the aspartic acid at position 6 also results in some reduction in binding.

### EXAMPLE 3

### Location of Binding Footprint on Published gD Crystal Structures

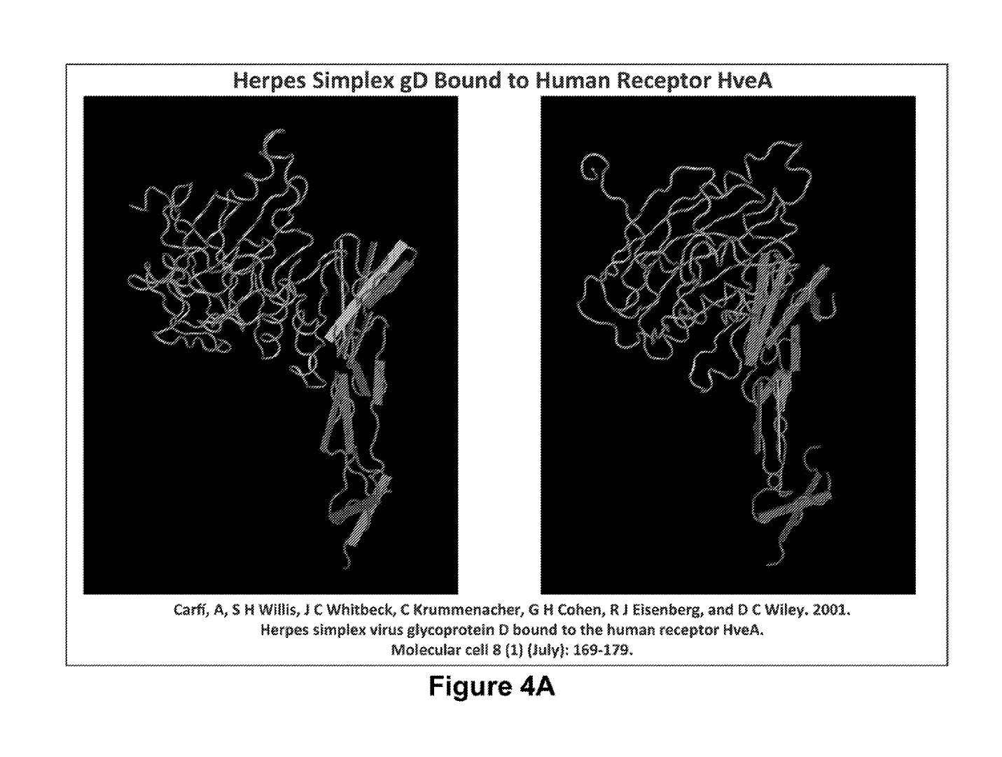

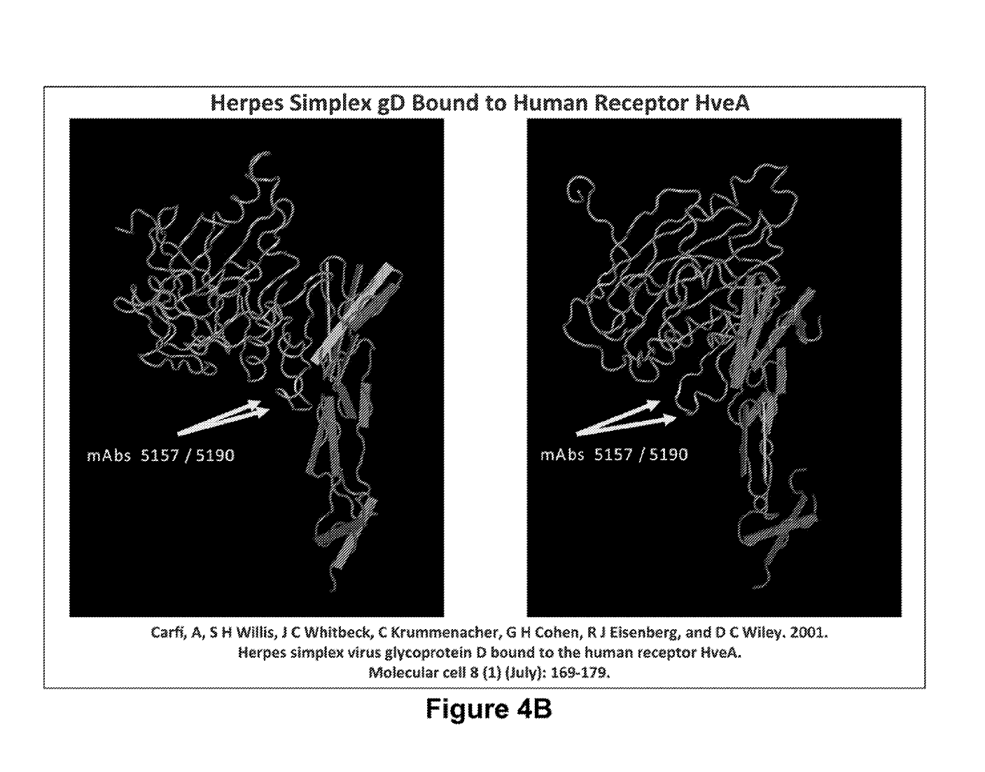

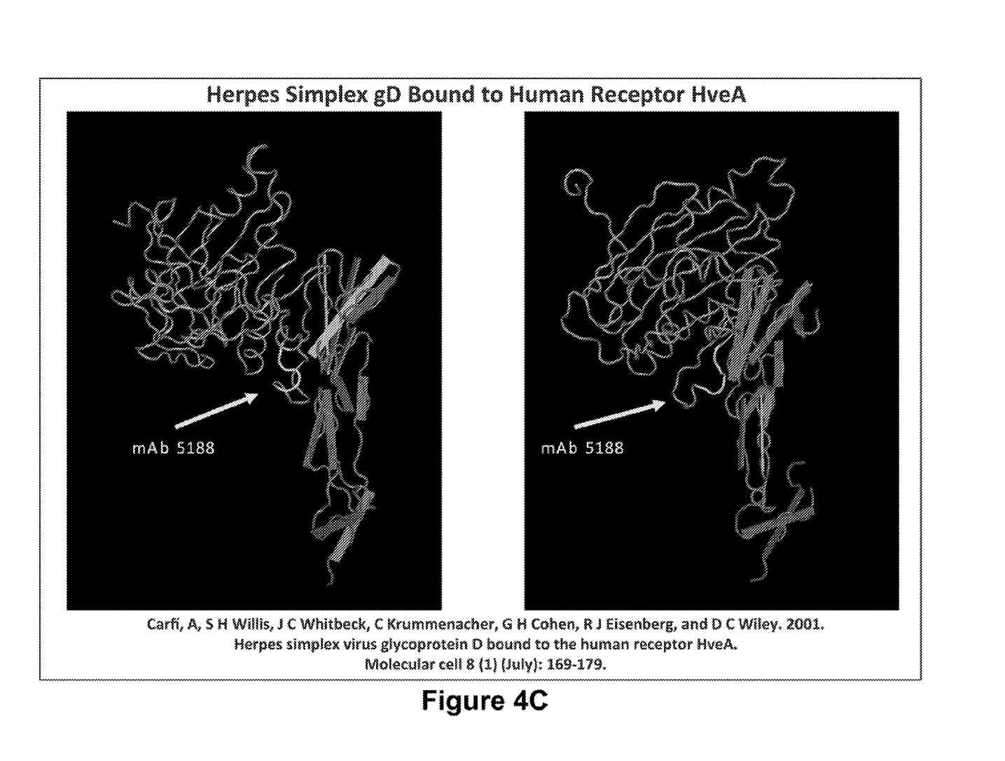

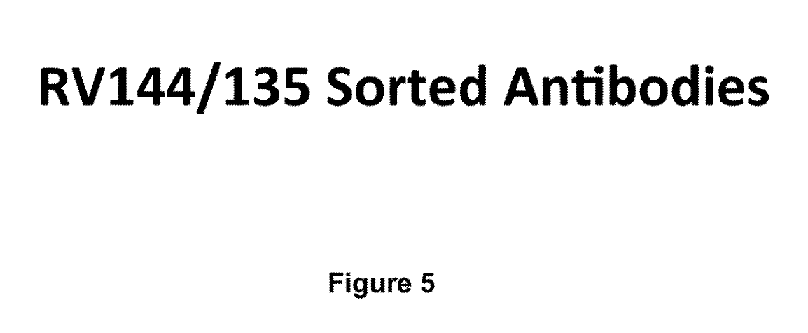

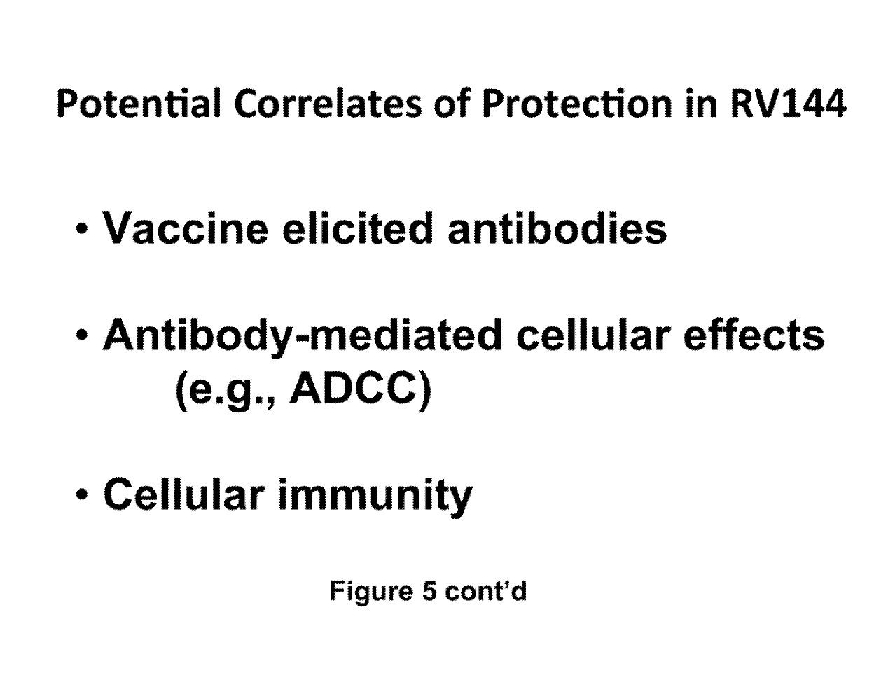

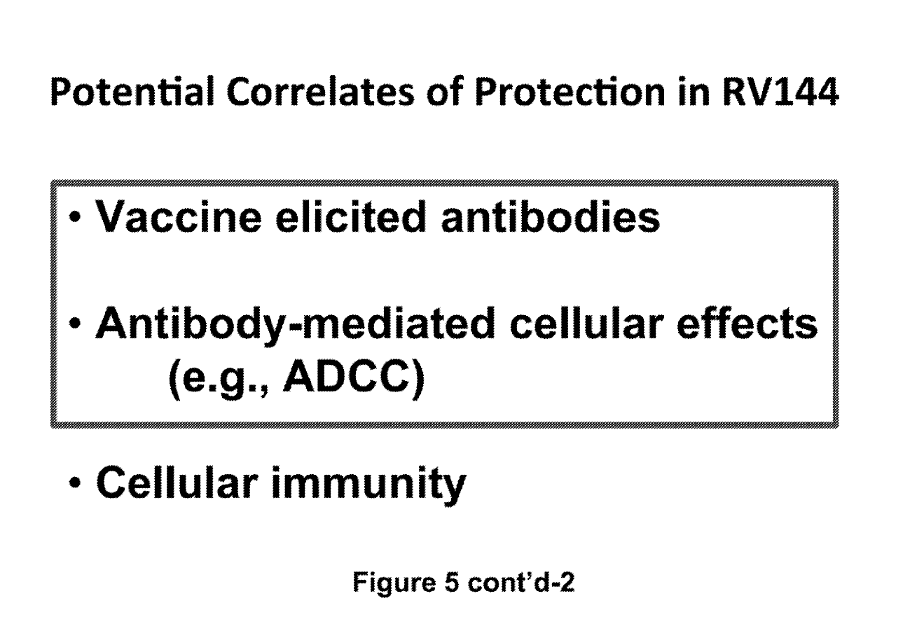

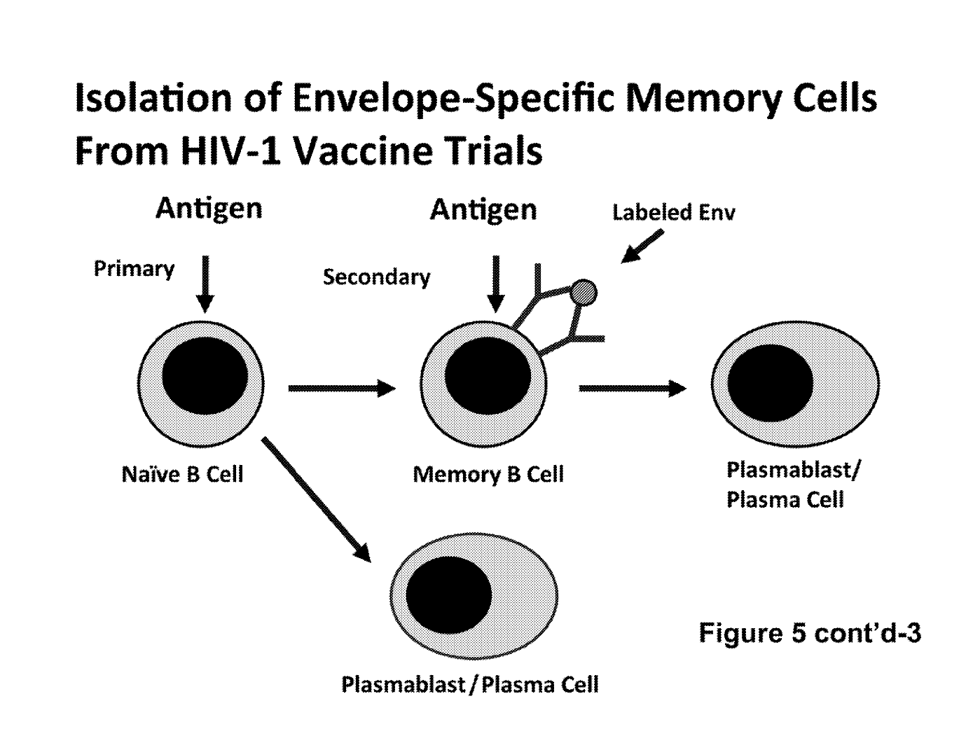

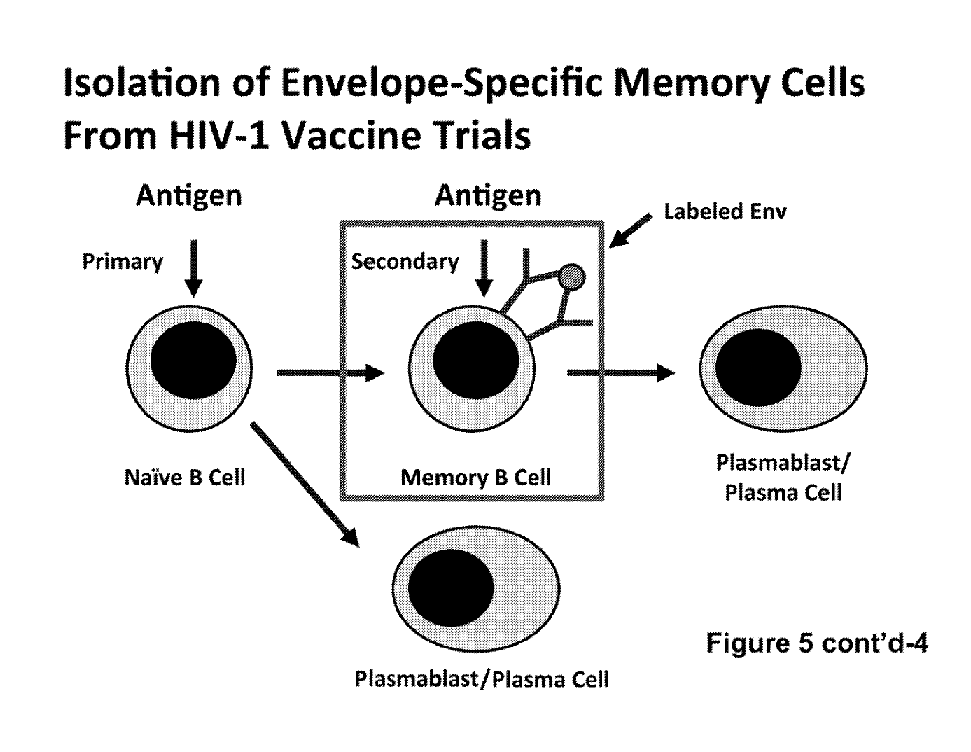

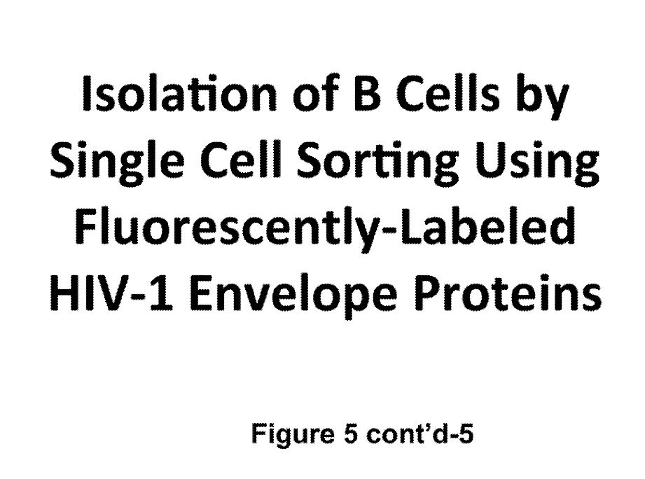

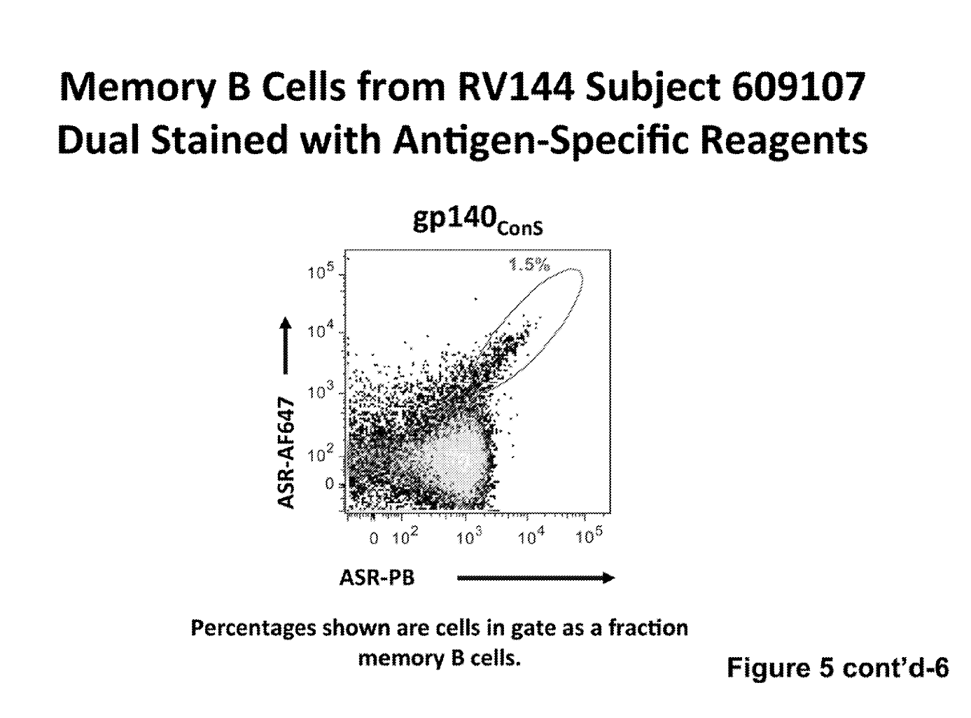

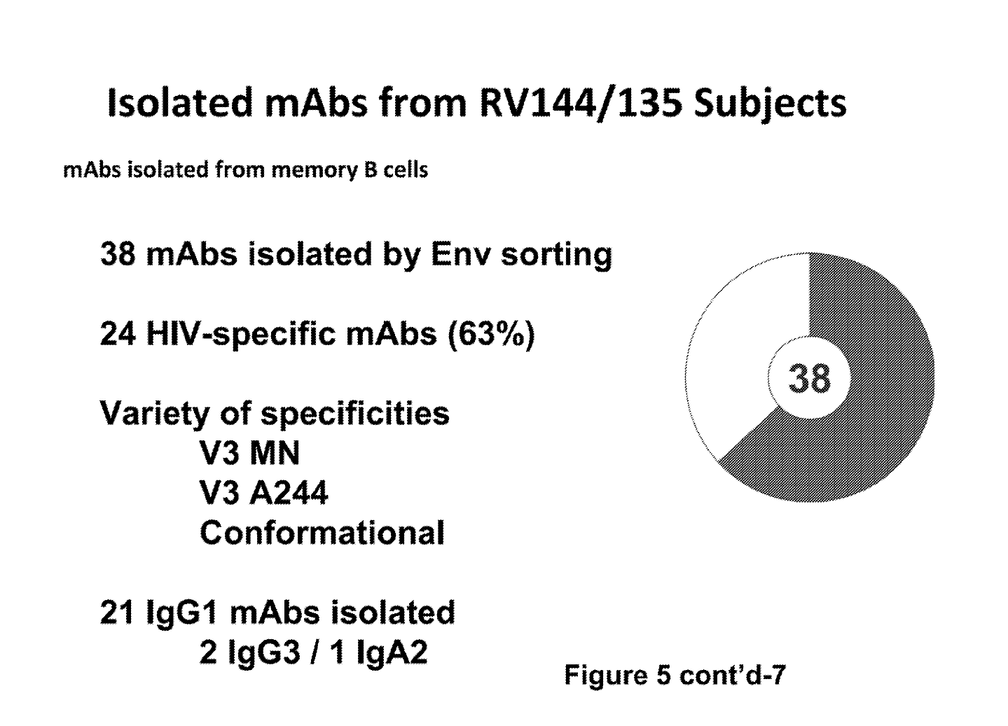

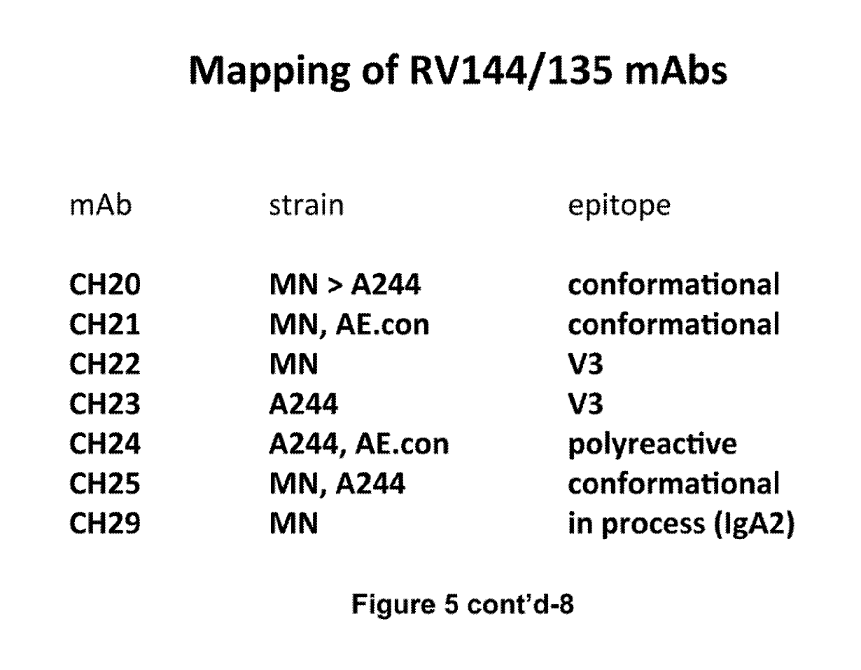

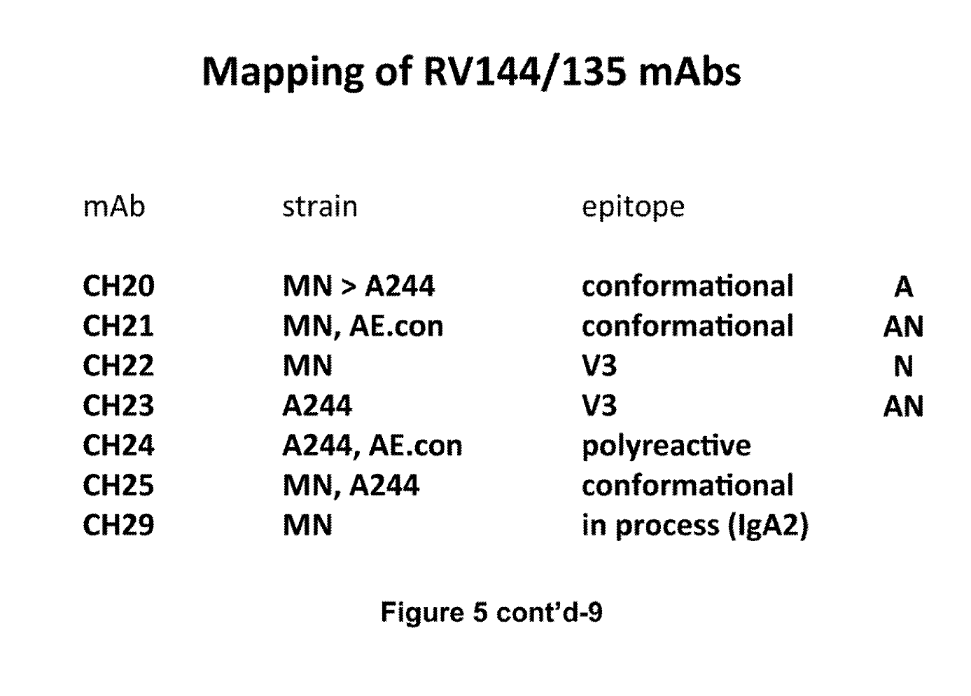

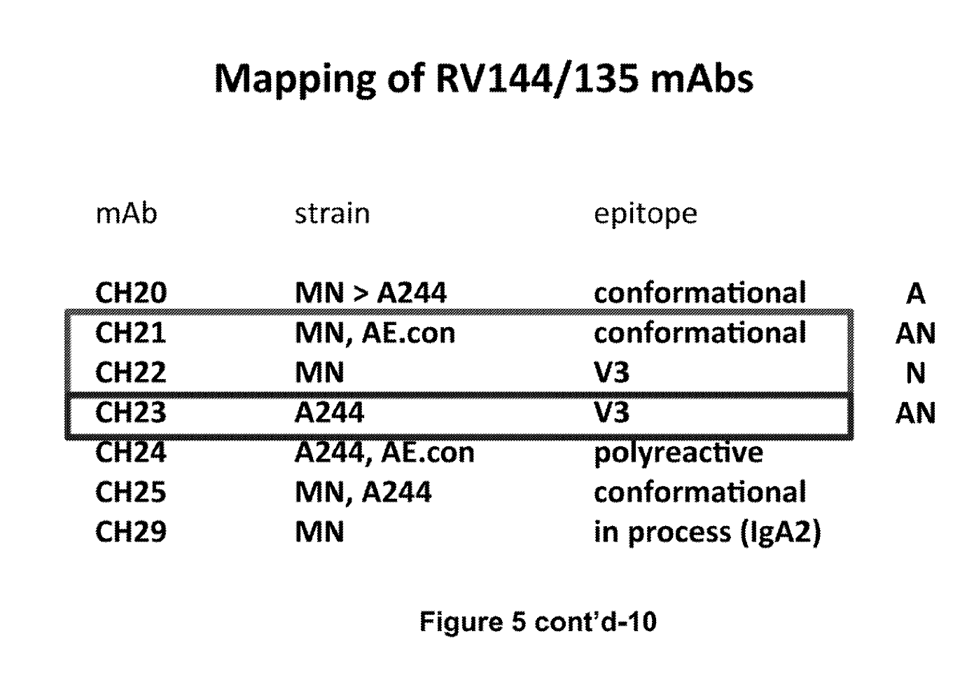

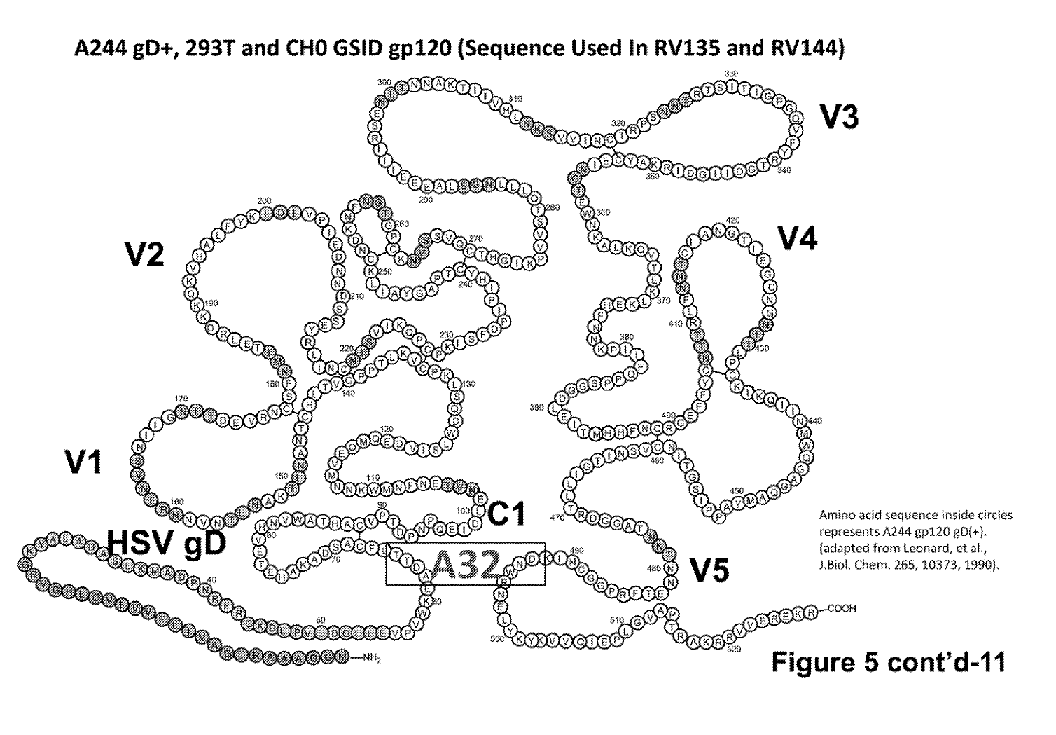

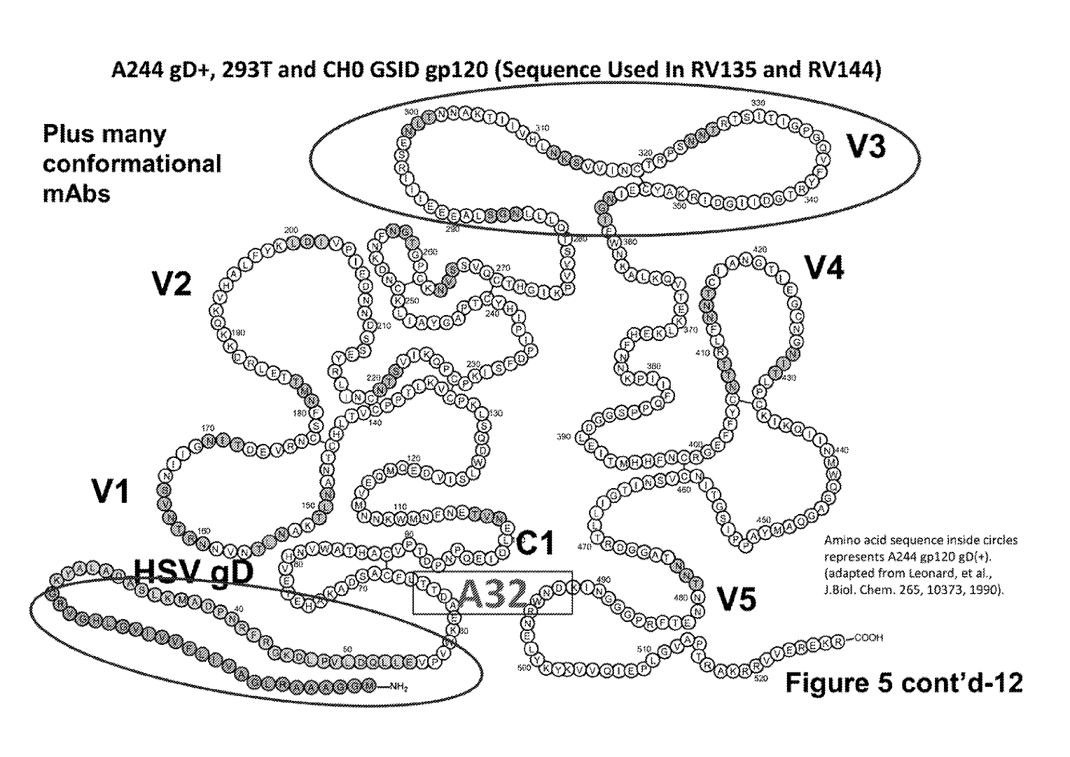

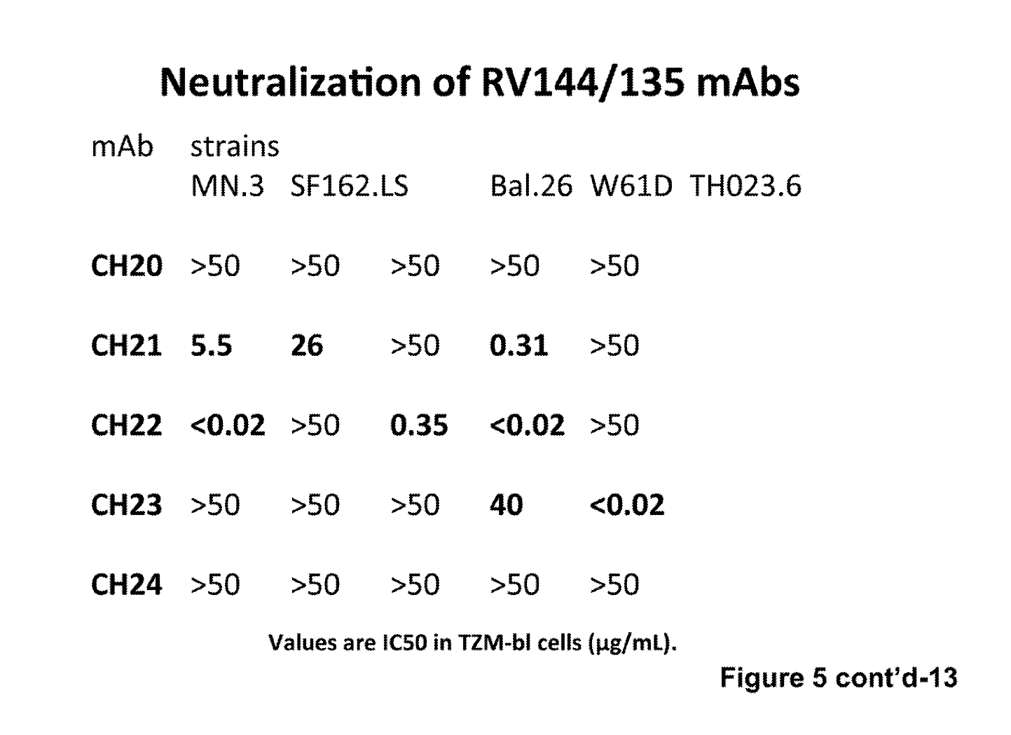

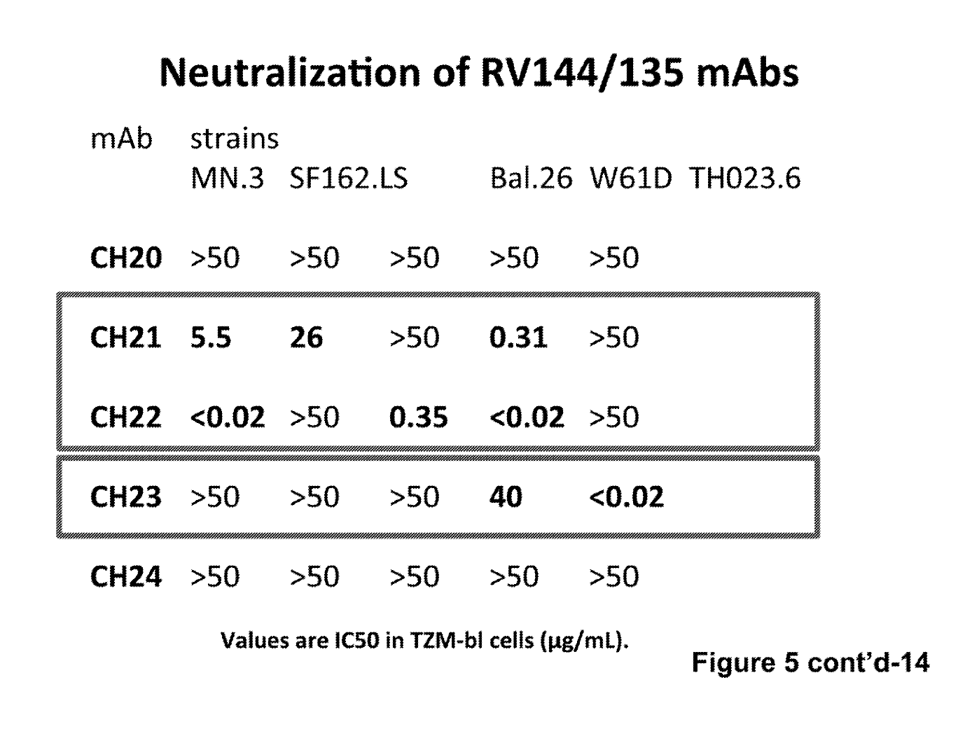

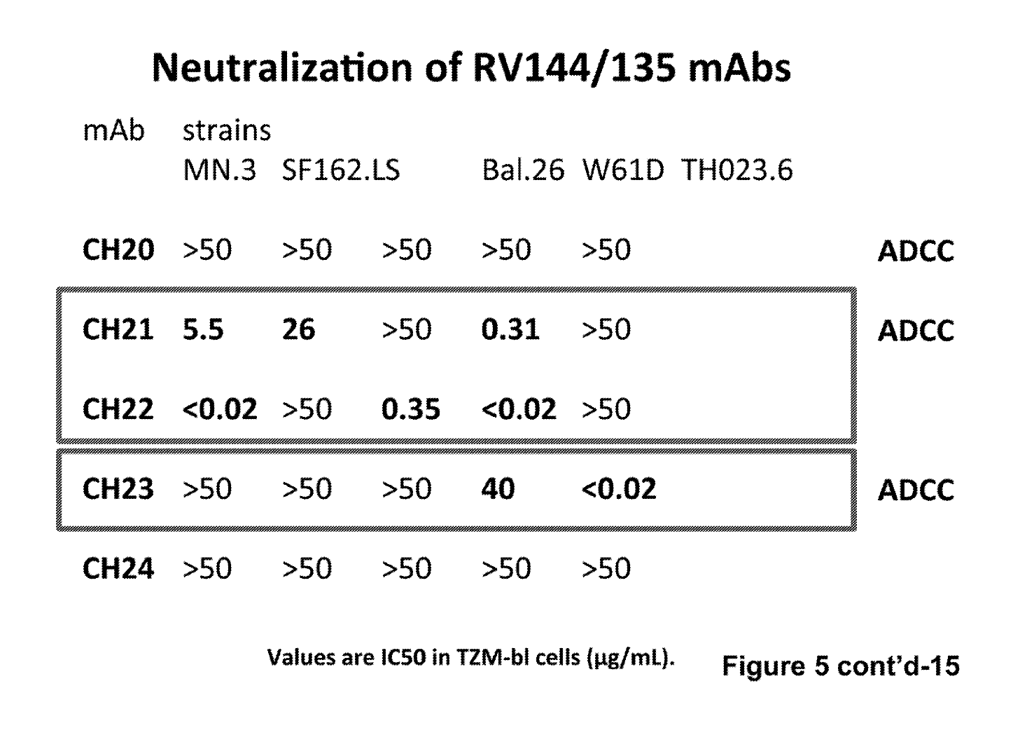

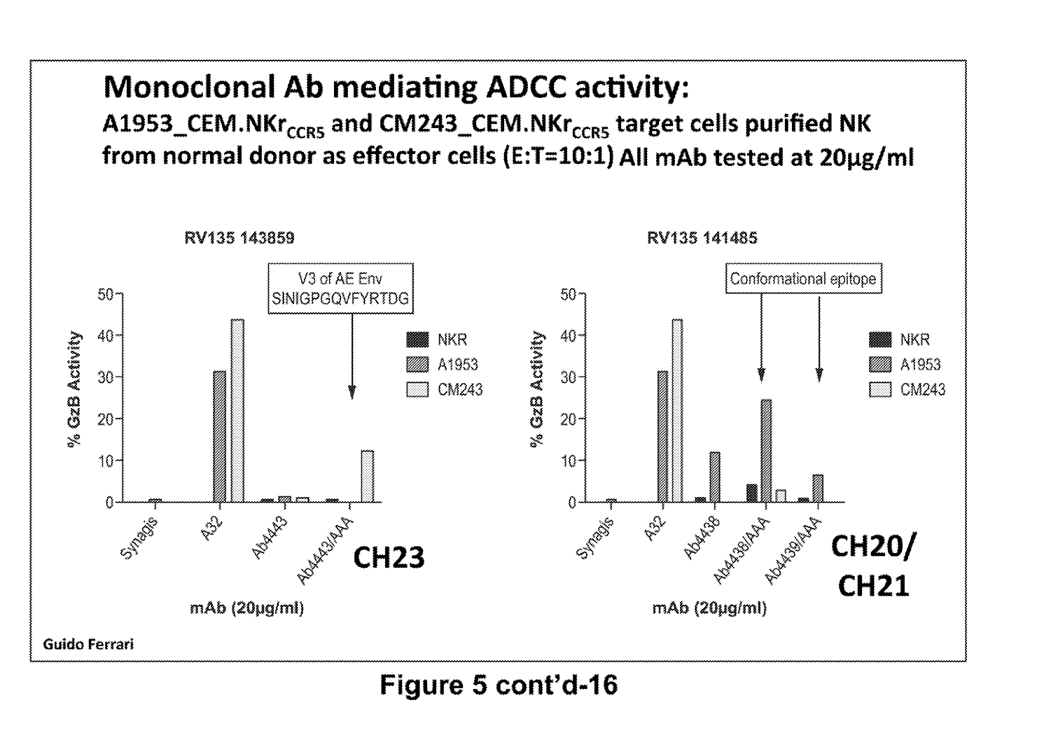

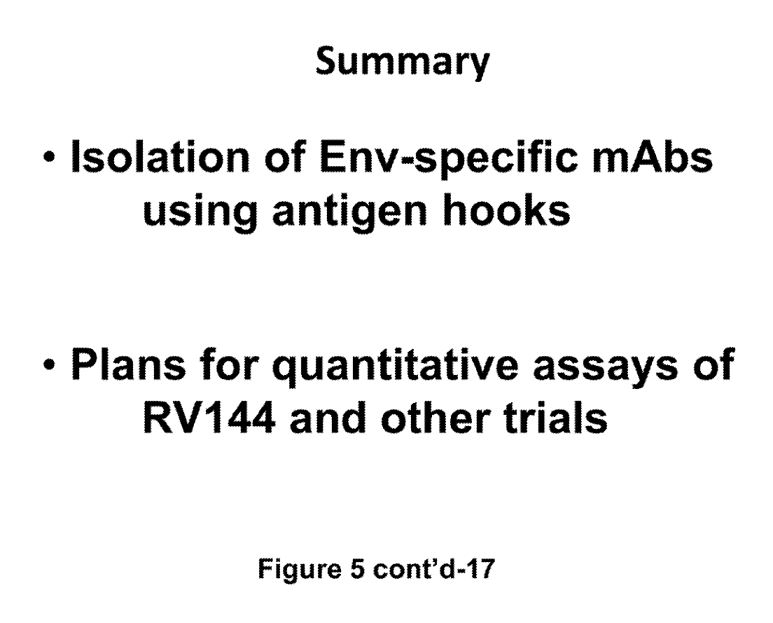

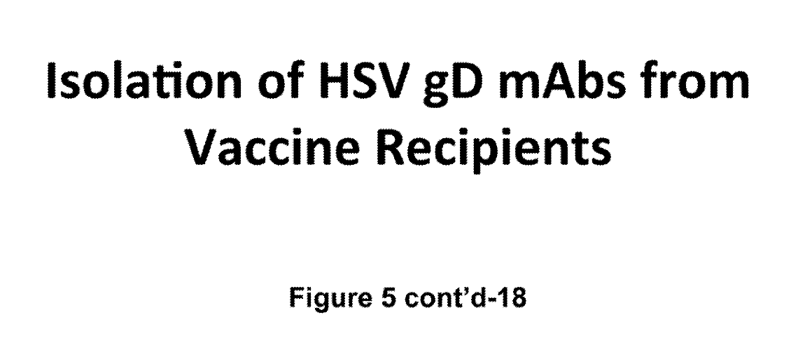

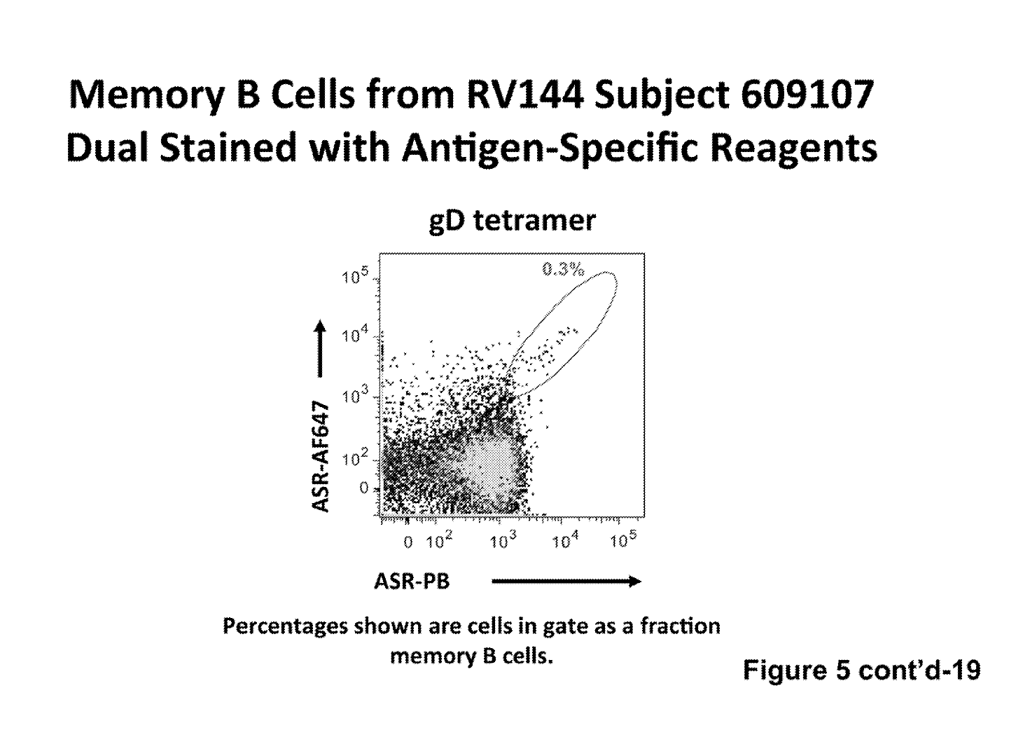

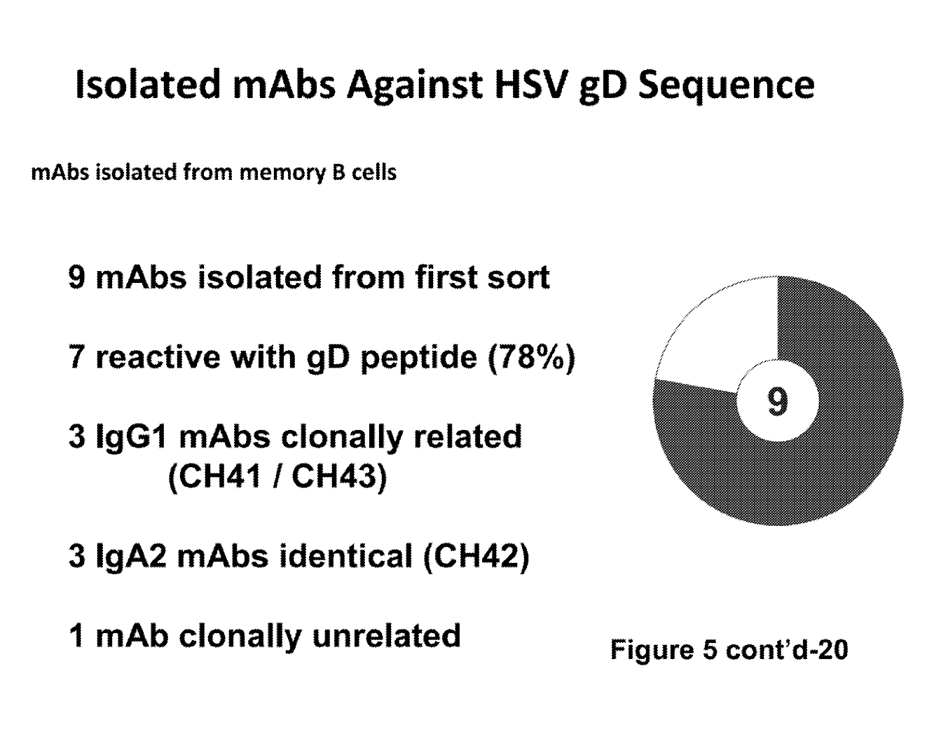

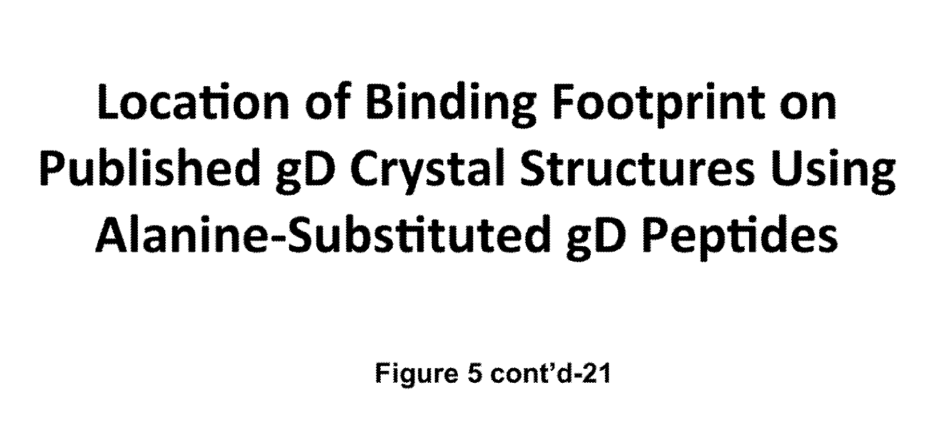

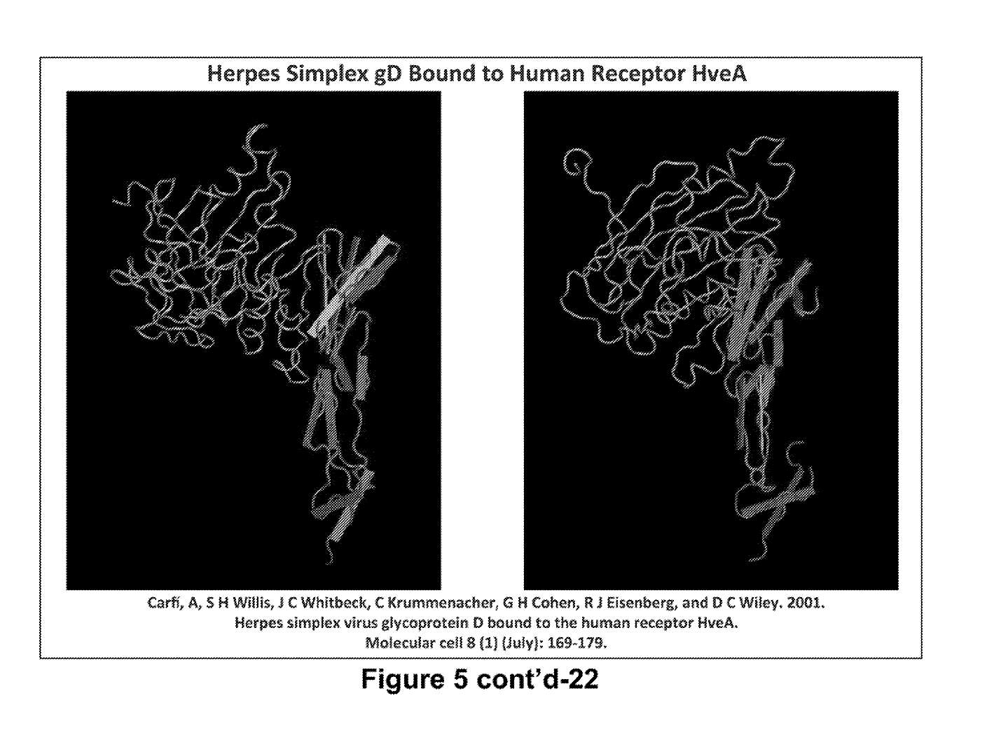

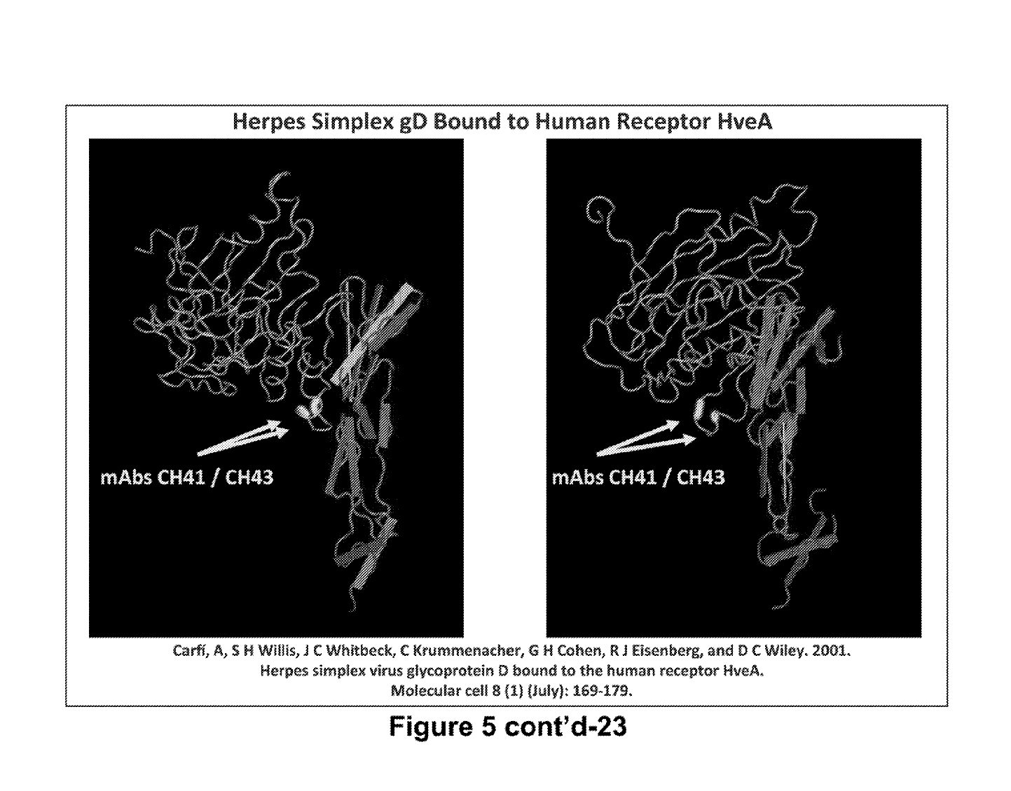

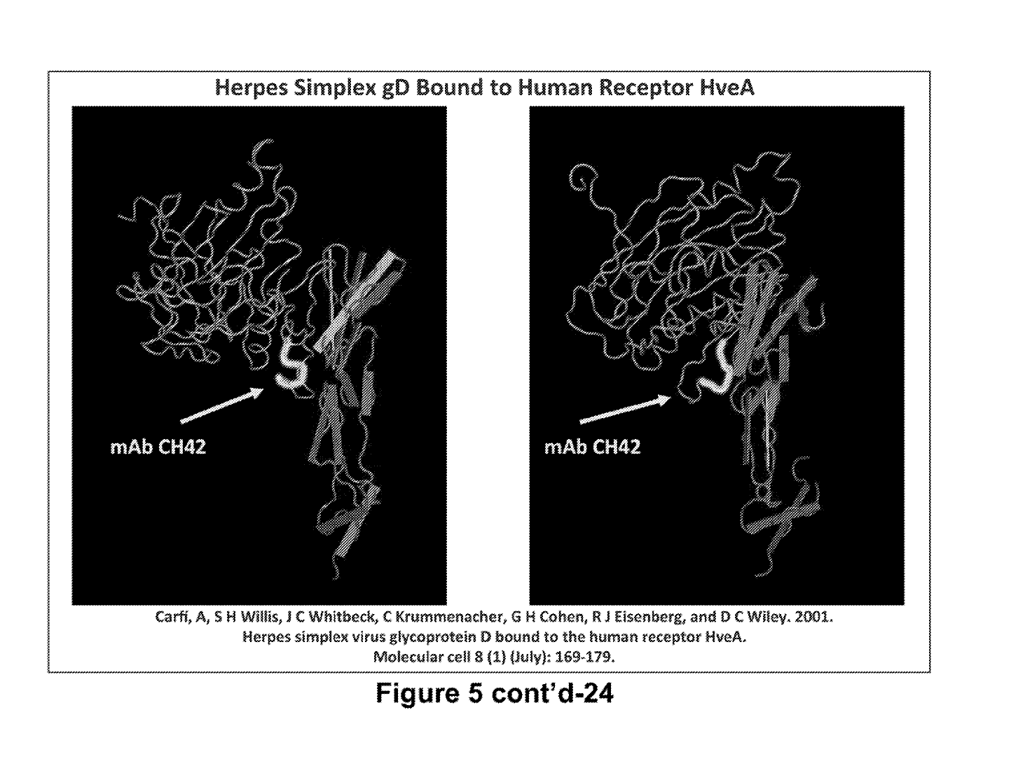

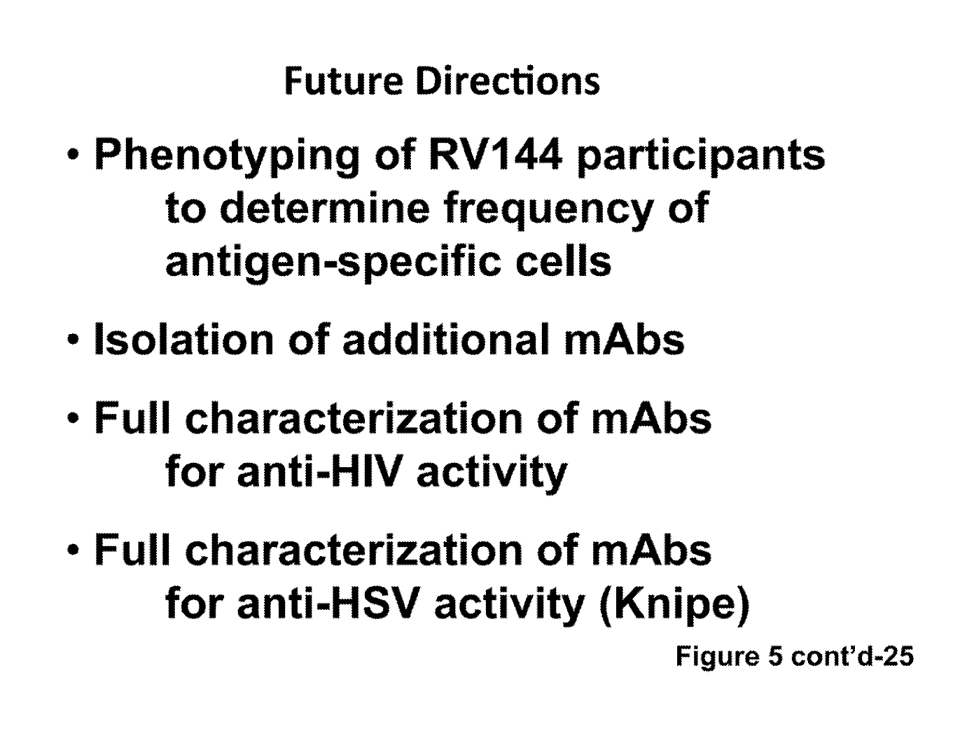

The crystal structure of the HSV gD protein complexed to one of its human receptors, HveA, is shown in FIG. 4A. The HSV gD protein is the globular protein shown in gray; HveA is shown in magenta and is to the right and slightly below HSV gD. Two views are shown, one slightly rotated compared to the other. The crystal structure was published by Carfi et al, (Molec. Cell 8 (1):169-179 (2001)).

Shown in FIG. 4B are the same views of the crystal structure shown in FIG. 4A with the two amino acids shown to be critical for binding (see FIGS. 3A and 3B) highlighted in yellow and pointed at by arrows. The residues critical for binding of mAbs 5157 (CH41) and 5190 (CH43) are near the contact points for gD-HveA interaction. The mAbs 5157 (CH41) and 5190 (CH43) would be expected to prevent binding of gD to its receptor.

Shown in FIG. 4C are the same views of the crystal structure shown in FIG. 4A with the amino acids shown to be critical for binding (see FIG. 3C) highlighted in yellow and pointed at by an arrow. The five residue sequence critical for mAb 5188 (CH42) binding is near the contact site for gD-HveA interaction and this mAb would also be expected to block binding of gD to its receptor.

All documents and other information sources cited above are hereby incorporated in their entirety by reference.

What is claimed is:

1. An isolated antibody specific for glycoprotein D (gD) of herpes simplex virus (HSV), or antigen binding fragment thereof.

2. The antibody according to claim 1 wherein said antibody comprises a complementarity determining region (CDR) of an antibody set forth in FIG. 2 or FIG. 6.

3. The antibody according to claim 1 wherein said antibody comprises a heavy or light chain amino acid sequence set forth in FIG. 2 or FIG. 6.

4. The antibody according to claim 1 wherein said antibody has the binding specificity of monoclonal antibody 5157, 5158, 5159; 5160; 5188, 5190, 5192 or an antibody set forth in FIG. 6.

5. An isolated nucleic acid comprising a nucleotide sequence encoding the antibody according to claim 1, or binding fragment thereof.

6. The nucleic acid according to claim 5 wherein said nucleic acid is present in a vector.

7. A method of preventing or treating HSV comprising administering to a subject in need thereof an antibody, or fragment thereof, according to claim 1 in an amount sufficient to effect said prevention or treatment.

8. The method according to claim 7 wherein said subject is a human.

9. The method according to claim 8 wherein said method is a method of preventing or treating HSV during pregnancy.

10. The method according to claim 8 wherein said human is immunocompromised.

11. A method of preventing or treating HSV comprising administering to a subject in need thereof said nucleic acid according to claim 5 under conditions such that said nucleotide sequence is expressed and said antibody, or fragment thereof, is produced in an amount sufficient to effect said prevention or treatment.

12. A composition comprising the antibody, or fragment thereof, according to claim 1, or the nucleic acid according to claim 5, and a carrier.

13. The composition according to claim 12 wherein said composition is in a form suitable for injection.

14. The composition according to claim 12 wherein said composition is in the form of a cream or ointment.
